(19) United States
(12) Patent Application Publication
(10) Pub. No.: US 2024/0058288 A1
(43) Pub. Date: Feb. 22, 2024

(54) DRUG COCKTAIL FOR TREATMENT OF PARKINSON'S DISEASE, LEWY BODY DISEASE AND MULTIPLE SYSTEM ATROPHY

(71) Applicant: ALETA NEUROSCIENCE, LLC, MARLTON, NJ (US)

(72) Inventor: Alexander SHTILBANS, Englewood Cliffs, NJ (US)

(21) Appl. No.: 18/490,503

(22) Filed: Oct. 19, 2023

Related U.S. Application Data
(63) Continuation-in-part of application No. PCT/IB2022/053599, filed on Apr. 18, 2022.
(60) Provisional application No. 63/177,362, filed on Apr. 20, 2021.
(60) Provisional application No. 63/203,819, filed on Jul. 30, 2021.
(60) Provisional application No. 63/261,151, filed on Sep. 14, 2021.
(60) Provisional application No. 63/263,262, filed on Oct. 29, 2021.

(51) Int. Cl. A61K 31/192; A61K 31/517; A61K 31/137; A61K 31/4422; A61K 31/198; A61K 31/122; A61K 31/506; A61K 31/575; A61K 38/26; A61P 25/28

(52) U.S. Cl. CPC A61K 31/192; A61K 31/517; A61K 31/137; A61K 31/4422; A61K 31/198; A61K 31/122; A61K 31/506; A61K 31/575; A61K 38/26; A61P 25/28

(57) ABSTRACT

A method of treating and preventing a neurodegenerative disorder associated with misfolding of α-synuclein is provided in a patient at risk for a neurodegenerative disorder or having symptoms of a neurodegenerative disorder, comprising the administration of a combination of two or more drugs selected from a chemical chaperone class of drugs, glycolysis enhancer class of drugs, a bile acid class of drugs, a glucagon-like-peptide-1 agonist (GLP-1) class of drugs, a glucocerebrosidase (GCase) inducer class of drugs, an iron chelator class of drugs, a mitochondrial antioxidant class of drugs, and a cluster-Abelson (c-Abl) tyrosine kinase inhibitor class of drugs. Specifically, the combination of drugs may include two or more drugs selected from sodium phenylbutyrate (PBA), tauroursodeoxycholic acid (TUDCA), exenatide (EXD), deferiprone (DFP), terazosin (TZ), creatine (CR), CoQ10, Ambroxol (AMB), and nilotinib (NL). The PBA may be provided in an extended-release formulation.

### FIELD OF THE INVENTION

Compositions to treat and prevent Parkinson's disease and related synucleinopathies are disclosed.

### BACKGROUND

Parkinson's disease (PD) is a common neurodegenerative disorder characterized by the progressive loss of dopaminergic neurons (dopaminergic neurodegeneration) of the substantia nigra pars compacta.(1) This dopaminergic neurodegeneration and subsequent loss of dopamine has serious consequences in the balance of the pathways of the basal ganglia. PD results in rigidity, tremor, slowness and postural instability and pathological findings of Lewy body deposition in the brain containing α-synuclein (alpha-synuclein) which is believed to contribute to dopaminergic cell death. There are several processes leading to neurodegeneration in Parkinson disease and other synucleinopathies such as Diffuse Lewy body Disease (DLBD) and Multiple System Atrophy (MSA). Those include: misfolding of alpha-synuclein which fails to be cleared from the brain, calcium excitotoxicity affecting mitochondria which results in energy depletion leading to neurodegeneration. Also, iron accumulation occurs in substantia nigra of the brain in patients with Parkinsonism which leads to activation of microglia and neuro inflammation which leads to oxidative stress, formation of reactive oxygen species (ROS) and ultimately to neurodegeneration as depicted in FIG. 1. This is an over-simplistic model of neurodegeneration in Parkinson disease as in reality it is believed that these processes take place in each patient at different times and different rates. Therefore, it is difficult to test a single medication aimed at improving just one of these processes in a clinical trial, because we do not know that all the participants have the same degree of dysfunction in the particular process which is studied.

A potential solution to this would be to either use personalized medicine where patients are identified with predominantly dysfunctional processes leading to neurodegeneration and identifying drugs to repair that dysfunction. Alternatively, a “cocktail” of different medications should be used which contains individual drugs aimed to address each of the above-mentioned processes which leads to neurodegeneration in general population of PD patients. This appears to be more realistic as it could be applied to all the patients suffering from Parkinson disease and possibly DLBD and MSA. Several medications have been tried individually in the past to slow down disease progression but failed as they only targeted one specific part of the complex Neurodegenerative process which I believe was not enough to show meaningful clinical effect. Moreover, the diversity and individual predominance of different parts of neurodegenerative chain in each individual patient made it impossible to discern potential improvement in the heterogeneous groups of patients evaluated in those studies.

Diffuse Lewy body Disease (DLBD) and Multiple System Atrophy (MSA) are other neurodegenerative disorders, atypical Parkinsonian subtypes, in which misfolded alpha-synuclein is similarly believed to contribute to neurodegeneration.

Unfortunately, neuroprotective efficacy in treating α-synucleinopathic diseases has been elusive. Existing medications only can provide improvement in motor symptoms and require increased doses as the disease progresses causing side effects. There are no known drugs that can stop, slow or reverse the disease progression.

### SUMMARY OF THE INVENTION

In an embodiment, a method is provided for treating a neurodegenerative disorder in a patient at risk for a neurodegenerative disorder associated with misfolding of α-synuclein or having symptoms of a neurodegenerative disorder associated with misfolding of α-synuclein. The method includes administering to the patient a combination of two or more drugs selected from a chemical chaperone class of drugs, a glycolysis enhancer class of drugs, a glucagon-like-peptide-1 agonist (GLP-1) class of drugs, a glucocerebrosidase (GCase) inducer class of drugs, an iron chelator class of drugs, a mitochondrial antioxidant class of drugs, a cluster-Abelson (c-Abl) tyrosine kinase inhibitor class of drugs and a bile acid class of drugs. In an embodiment, the method as described in this paragraph includes administering to the patient a combination of three or more drugs from the same group of drugs.

In an embodiment, the chemical/molecular chaperone class of drugs includes one sodium phenylbutyrate (PBA) and arimoclomol. In an embodiment, the bile acid class of drugs may be one or more of tauroursodeoxycholic acid (TUDCA), ursodeoxycholic acid (UDCA) and deoxycholic acid (DCA). In an embodiment, the glycolysis enhancer may be terazosin (TZ). In an embodiment, the GLP-1 class of drugs may be one or more of Exenatide, ORMD-0901, dulaglutide, demaglutide, liraglutide, or lixisenatide. In an embodiment, the GCase inducer class of drugs may include ambroxol (AMB), BIA 28-5156 (LTI-291); isofagomine; LB-205 and S-181. In an embodiment, the iron chelator class of drugs may include a drug selected from deferiprone (DFP), deferoxamine (DFO), desferrioxamine, deferasirox, clioquinol, tetrahydrosalen, 5,7-Dichloro-2-[(dimethylamino)methyl]quinolin-8-ol (PBT2), (N,N,N,N-Tetrakis(2-pyridylmethyl)-ethylenedi-amine) (TPEN), 1,10-phenanthroline (PHEN), 1,2-hydroxypyridinone (1,2-HOPO), clioquinol; 5-[N-methyl-N-propargylaminomethyl]-8-hydroxyquinoline dihydrochloride (M30); M31; M32; -[4-(2-hydroxyethyl)piperazine-1-ylmethyl]-quinoline-8-ol] (VK28), HLA16, HLA20, M32, M10, SIH-B, BSIH, pyridoxal isonicotinoyl hydrazine (PIH); 2-pyridylcarboxaldehyde isonicotinoyl hydrazine (PCIH), H2NPH, and H2PPH or a combination thereof. In an embodiment, the mitochondrial antioxidant class of drugs may include creatinine or CoQ10. In an embodiment, the c-Abl tyrosine kinase inhibitor class of drugs may include a drug selected from nilotinib radotinib, vodobatinib (K0706), bafetinib, imatinib, dasatinib, bosutinib, ponatinib, rebastinib, tozasertib, and danusertib or a combination thereof.

In an embodiment, the PBA may be provided in an extended-release formulation.

In an embodiment, the neurogenerative disorder is an alpha-synucleinopathy selected from Parkinson's disease (PD), Diffuse Lewy body Disease (DLBD) and Multiple System Atrophy (MSA).

In an embodiment, a method is provided for treating a neurodegenerative disorder associated with misfolding of α-synuclein comprising the administration, to a patient at risk for a neurodegenerative disorder or having symptoms of a neurodegenerative disorder, wherein the method includes the administration of a combination of two or more drugs selected from sodium phenylbutyrate (PBA), tauroursodeoxycholic acid (TUDCA), exenatide (EXD), deferiprone (DFP), terazosin (TZ), creatine (CR), CoQ10, Ambroxol (AMB), and nilotinib (NL). In an embodiment, the method of this paragraph includes a combination comprises three or more drugs selected from the same list of drugs.

In an embodiment, the method provides a combination of sodium phenylbutyrate (PBA) and exenatide. In an embodiment, the method provides a combination of sodium phenylbutyrate (PBA), tauroursodeoxycholic acid (TUDCA), and exenatide (EXD). In an embodiment, the method provides a combination of NL and TUDCA. In an embodiment, the method provides a combination of PBA, CR, and CoQ10. In an embodiment, the method provides a combination of EXD and TUDCA. In an embodiment, the method provides a combination of PBA, EXD, and AMB. In an embodiment, the method provides a combination of EXD, NL, and TUDCA. In an embodiment, the method provides a combination of PBA, EXD, DFP. In an embodiment, the method provides a combination of PBA, NL, and TUDCA. In an embodiment, the method provides a combination of EXD and NL. In an embodiment, the method provides a combination of PBA, EXD, and NL. In an embodiment, the method provides a combination of PBA, EXD, TUDCA, and DFP.

In an embodiment, the neurodegenerative disorder is Multiple System Atrophy (MSA) and the drugs administered are sodium phenylbutyrate (PBA) and tauroursodeoxycholic acid (TUDCA).

### DESCRIPTION OF THE DRAWINGS

FIG. 1. Diagram showing different proposed processes leading to dopaminergic neurodegeneration in Parkinson's Disease. Numbers in parentheses refer to drugs from Table 1 which inhibit that particular process. Abbreviation key: PBA: Sodium Phenylbutyrate; TZ: terazosin; EXD: Exenatide; AMB: Ambroxol; DFP: Deferiprone, CR: creatine; CoQ10: Coenzyme Q10; NL: Nilotinib; TUDCA: tauroursodeoxycholic acid.

FIG. 1 FIG. 2A is a plot of neurite length (mm/mm²) vs. time of controls plus experimental combinations 1-16.

FIG. 2B is a plot of neurite branching (mm²) vs. time of controls plus experimental combinations 1-16.

FIG. 3A is bar plot summarizing the data for neurite length (mm/mm²) of controls plus experimental combinations 1-16.

FIG. 3B is a bar plot summarizing the data for branching (mm²) of controls plus experimental combinations 1-16.

FIG. 4A is a plot of raw data of neurite length (mm/mm²) vs. time of controls plus experimental combinations 17-28.

FIG. 4B is a plot of raw data of neurite branching (mm²) vs. time of controls plus experimental combinations 17-28.

FIG. 5A is bar plot summarizing the data for neurite length (mm/mm²) of controls plus experimental combinations 17-28.

FIG. 5B is a bar plot summarizing the data for branching (mm²) of controls plus experimental combinations 17-28.

FIG. 6A is bar plot summarizing the data for neurite length (mm/mm²) of controls plus experimental combinations 10, 12, 51-53.

FIG. 6B is a bar plot summarizing the data for branching (mm²) of controls plus experimental combinations 10, 12, 51-53.

FIG. 6C is a bar plot summarizing the data for cytolysis (%) of controls plus experimental combinations 10, 12, 51-53.

FIG. 7A is bar plot summarizing the data for neurite length (mm/mm²) of controls plus experimental combinations 23, 24, 29-41.

FIG. 7B is a bar plot summarizing the data for branching (mm²) of controls plus experimental combinations 23, 24, 29-41.

FIG. 7C is a bar plot summarizing the data for cytolysis (%) of controls plus experimental combinations 23, 24, 29-41.

FIG. 8. Shows a graphic representation of data in Table 10.1 showing inhibition of IL-6 induced neuroinflammation by various test conditions of PBA, TUDCA, and EXD.

FIG. 9. Shows a graphic representation of data in Table 10.2 showing inhibition of TNF-α induced neuroinflammation by various test conditions of PBA, TUDCA, and EXD.

FIG. 10. Shows a graphic representation of data in Table 10.3 showing inhibition of CXCL1/GROα induced neuroinflammation by various test conditions of PBA, TUDCA, and EXD.

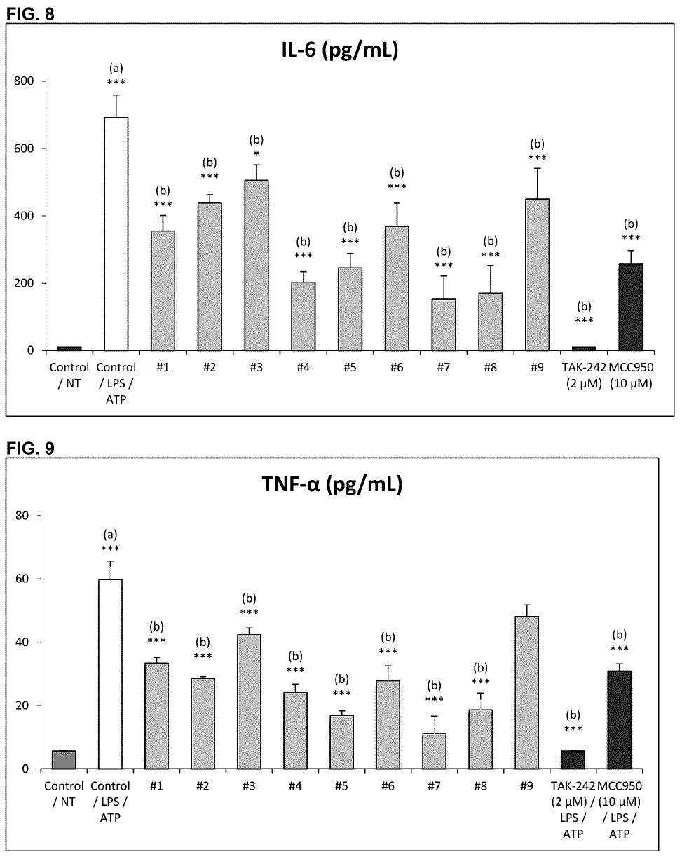

Statistical analysis on FIGS. 8-10: Intergroup comparison: no asterisk=Not Significant; *=p<0.05; **=p<0.01; ***=p<0.001. (a): compared with Control/NT (untreated microglia); (b): compared with Control/LPS/ATP (activated microglia).

### DETAILED DESCRIPTION

Disclosed herein are combinations of drugs designed to treat or prevent of neurogenerative disorders related to misfolding of α-synuclein (also termed herein alpha-synuclein) including Parkinson's disease (PD), Diffuse Lewy body Disease (DLBD) and Multiple System Atrophy (MSA). This family of diseases is associated with the progressive loss of dopaminergic neurons (dopaminergic neurodegeneration) of the substantia nigra pars compacta. The substantia nigra pars compacta is a cluster of cells in the brain that releases the neurotransmitter dopamine to neurons of the striatum which project to the basal ganglia. The basal ganglia is connected to the thalamus and motor cortex which imparts control over motor output.(2)

In an embodiment, a method is provided for treating a neurodegenerative disorder in a patient at risk for a neurodegenerative disorder or having symptoms of a neurodegenerative disorder, by the administration of a combination of two or more drugs selected from a chemical chaperone class of drugs, glycolysis enhancer class of drugs, a glucagon-like-peptide-1 agonist (GLP-1) class of drugs, a glucocerebrosidase (GCase) inducer class of drugs, an iron chelator class of drugs, a mitochondrial antioxidant class of drugs, a cluster-Abelson (c-Abl) tyrosine kinase inhibitor class of drugs and a bile acid class of drugs. The combination of drugs may include two or more drugs selected from this list or three or more drugs selected from this list.

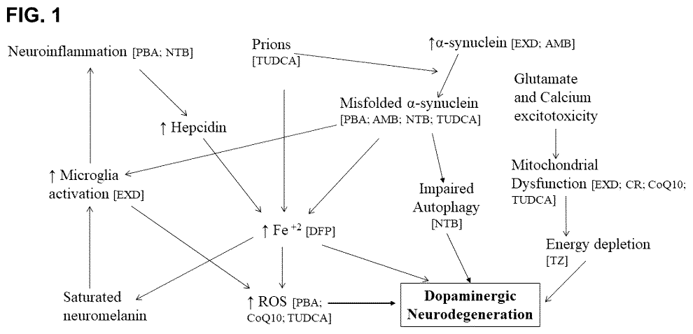

A number of specific processes are implicated in dopaminergic neurodegenerative diseases. FIG. 1 is a diagram showing relationships believed to lead to dopaminergic neurodegeneration in Parkinson's Disease. Starting from the top right of FIG. 1, overaccumulation and misfolding of α-synuclein in the brain coupled with impaired autophagy leads to dopaminergic degeneration. Moreover, misfolded α-synuclein leads to over-accumulation of intracellular iron(II) in the substantia nigra of the brain which in turn leads to formation of reactive oxygen species (ROS) and neurodegeneration (3), (4). The over-accumulated iron is removed from the brain by the natural iron chelator neuromelanin (5), (6). Once neuromelanin is saturated with iron, it causes activation of microglia which leads to formation of more ROS as well as neuroinflammation which in turn results in more brain iron accumulation closing the feedback loop (7).

Prion-like cell-to-cell disease transmission may also be a significant factor in many neurodegenerative disorders. Prions have been associated with iron dyshomeostasis(8) and misfolding of α-synuclein leading to neurodegeneration.(9)

The codes in brackets refer to drugs from Table 1 which inhibit that particular process. The codes are: PBA: Sodium Phenylbutyrate; TZ: terazosin; EXD: Exenatide; AMB: Ambroxol; DFP: Deferiprone, CR: creatine; CoQ10: Coenzyme Q10; NL: Nilotinib; TUDCA: tauroursodeoxycholic acid. For example, activation of microglia can lead to neuroinflammation and neurodegeneration, and exenatide (EXD in Table 1) inhibits activation of microglia. All of these drugs are known individually, and many are known or suspected to have some degree of activity in the treatment of neurodegenerative disorders based on preclinical studies. However, the combination of these drugs as illustrated by the flow chart of FIG. 1 may have additive/synergistic and enhanced activity over any individual drugs. In embodiments, two or more of these drugs may be used to prevent or treat neurodegenerative disorders caused by misfolding of α-synuclein, mitochondrial dysfunction and neuroinflammation.

The drugs discussed herein can be classified into distinct mechanistic classes (also termed groups), as discussed below, including chemical chaperones, glycolysis enhancers, GLP-1 agonists, GCase inducers, iron chelators, mitochondrial enhancers, c-Abl tyrosine kinase inhibitors and bile acids.

In an embodiment, this invention discloses a combination of two or more drugs selected from a chemical chaperone class of drugs, glycolysis enhancer class of drugs, a glucagon-like-peptide-1 agonist (GLP-1) class of drugs, a glucocerebrosidase (GCase) inducer class of drugs, an iron chelator class of drugs, a mitochondrial antioxidant class of drugs, a cluster-Abelson (c-Abl) tyrosine kinase inhibitor class of drugs and a bile acid class of drugs.

In an embodiment, this invention discloses a combination of drugs from the list on Table 1. In an embodiment, this invention discloses at least two drugs in combination. In an embodiment, this invention discloses a combination of at least two drugs wherein the drugs are selected from at least two different classes of drugs.

Table 1 lists compounds studied in this invention. These drugs have been studied individually in the past for neurodegenerative disorders. This invention proposes to use various combinations of the compounds in Table 1, which are postulated to stop or slow down individual parts of the neurodegenerative processes seen in PD, MSA and DLBD which we expect can produce additive or synergistic effects which are expected to show clinical efficacy in human trials.

Chemical Chaperones

Sodium 4-phenylbutyrate, (PBA) is an FDA-approved therapy for reducing plasma ammonia and glutamine in urea cycle disorders. PBA has anti-inflammatory activity, reduces ROS and misfolded α-synuclein. This drug is a histone deacetylase inhibitor and can suppress both proinflammatory molecules and reactive oxygen species in activated glial cells in the brain (10). It also acts as a chemical chaperone and can prevent aggregation of misfolded α-synuclein and suppress endoplasmic reticulum stress (11). Previous preclinical studies showed that it halted the disease progression in chronic PD and DLBD mouse models and may be of therapeutic benefit for PD (10) and possibly MSA and DLBD as well.

Sodium 4-phenylbutyrate is termed a “hydrophobic chemical chaperone.” The general mechanism of action proposed for chemical chaperones involves the interaction of hydrophobic regions of the chaperone with exposed hydrophobic segments of the unfolded protein. This interaction protects the protein from aggregation. Other compounds studied as chemical chaperones include deoxycholic acid (DCA), ursodeoxycholic acid (UDCA), and tauroursodeoxycholic acid (TUDCA). A related series of compounds are osmolyte chemical chaperones, which include free amino acids and amino acid derivatives (e.g., glycine, taurine, β-alanine), polyols (e.g., glycerol, sucrose), methyl-amines (e.g., trimethylamine N-oxide (TMAO)), and dimethyl sulfoxide (DMSO). Another histone deacetylase inhibitor is arimoclomol which is also a molecular chaperone. Under stressful conditions in a cell, it induces heat-shock protein aimed to correct misfolding of other proteins.

A drawback to the therapeutic use of PBA in humans is the high dosage requirement, up to 15 g/day. In an embodiment, it may be possible to reduce this dosage by the use of an extended release formulation as disclosed by Truog (12). For example, PBA can be formulated with a hydrophilic polymer. The hydrophilic polymer may be at least one cellulose ether polymer selected from the group consisting of methylcellulose, hydroxyethyl cellulose and hydroxypropyl cellulose. The hydrophilic polymer may be selected from the group consisting of non-cellulose polysaccharides, polyethylene oxide, polyvinyl alcohols and acrylic acid co-polymers. In another example, a PBA extended-release formulation can be an osmotic device, which is a tablet having a core of an active ingredient combined with an osmotic agent. The tablet may be coated with a semipermeable membrane that allows water to pass through the membrane into the core but not out of the membrane. The water that enters the tablet elevates the osmotic pressure from the osmotic agent inside the coated tablet. An orifice in the tablet relieves the pressure and allows the active agent to flow out of the tablet at a controlled rate. Other extended or controlled release systems are possible. As used herein, the term “extended release” is synonymous with “controlled release,” “sustained release,” and “modified release.”

The proposed dose of PBA for clinical use is up to 3 g orally twice daily.

Bile Acids

Bile acid drugs include tauroursodeoxycholic acid (TUDCA), deoxycholic acid (DCA), and ursodeoxycholic acid (UDCA). Tauroursodeoxycholic acid (TUDCA) is an endogenous bile acid and a chemical chaperone that functions as a strong neuroprotective agent that also acts as a chemical chaperone. TUDCA is a taurine conjugate of ursodeoxycholic acid (UDCA). It is permeable to the blood-brain barrier and has a low toxicity profile (13),(14). TUDCA has been shown to have beneficial effects in preclinical models of AD, ALS, PD and HD. These disorders share the common crucial feature of accumulation of misfolded protein aggregates in the brain (15).

Bile acids such as UDCA and TUDCA, have been shown to suppress the toxic aggregation of misfolded proteins in various animal models of neurodegenerative diseases. These bile acids safeguard neurons also by reducing the synthesis of reactive oxygen species, mitigating mitochondrial damage, and inhibiting apoptosis through both the intrinsic and extrinsic pathways (13). Moreover, TUDCA and UDCA substantially reduced PrP conversion in cell-free aggregation assays, and in chronically and acutely infected cell cultures. TUDCA and UDCA also reduced neuronal loss in prion-infected cerebellar slice cultures suggesting they may have a therapeutic role in prion diseases.(16) Duan et al showed that the application of TUDCA facilitates the survival of DA neurons in vitro and in vivo conditions.(17) TUDCA-treated group demonstrated increase in the number of tyrosine hydroxylase positive neurons, used as a marker for dopamine, norepinephrine, and epinephrine-containing neurons; (18) and a reduction in the number of apoptotic cells.

In a mouse model of MPTP (1-methyl-4-phenyl-1,2,3,6-tetrahydropyridine; a prodrug of MPP+ that can be administered to animals) induced Parkinsonism, pre-treatment with TUDCA (50 mg/kg for 3 days) significantly reduced neurodegeneration of the nigral dopaminergic neurons caused by MPTP, as well as reduced dopaminergic fiber loss and ameliorated motor performance and symptoms typical of PD, such as spontaneous activity, ability to initiate movement and tremors. (19) TUDCA treatment also prevented the production of MPTP-dependent ROS in GSTP null mice.(19) TUDCA-dependent mitoprotective effects have also been observed in primary mouse cortical neurons and neuroblastoma cell line SH-SY5Y (20). All this makes TUDCA useful in attenuating mitochondrial dysfunction and ROS production as well as inhibiting multiple proteins involved in apoptosis.

Bile acids also block apoptotic pathways in dopamine neurons, prevents mitochondrial dysfunction, ROS production and neuroinflammation (21); antioxidant. Bile acids also have antiapoptotic activity for dopamine neurons. This prevents mitochondrial dysfunction, ROS production and neuroinflammation, as well as inhibiting multiple proteins involved in apoptosis.

In an embodiment, the neurodegenerative disorder is MSA and the drugs used to treat MSA are a combination of PBA and TUDCA. In other embodiments, this invention excludes a combination of PBA and TUDCA with no other drugs as disclosed herein.

A proposed dose of TUDCA for clinical use is up to 1 g orally twice daily. A proposed dose of UDCA for clinical use is up to 15 mg/kg twice daily.

Glycolysis Enhancer

Terazosin (TZ), an FDA approved therapy for hypertension and benign prostatic hyperplasia enhances the activity of phosphoglycerate kinase 1, thereby stimulating glycolysis and increasing cellular ATP levels. In previously published toxin-induced and genetic PD models in mice, rats, flies, and induced pluripotent stem cells, TZ increased brain ATP levels and slowed or prevented neuron loss. This drug also increased dopamine levels and partially restored motor function (22). Moreover, a recent retrospective population study showed that people taking terazosin had a lower hazard ratio to develop PD in their lifetime (23). A proposed dose for the clinical use is up to 5 mg orally at bedtime.

Glucagon-Like Peptide-1 (GLP-1) Agonists

Exenatide prevents microglia activation, reduces neuroinflammation, reduces accumulation of misfolded α-synuclein and improves mitochondrial function.

Exenatide (a synthetic version of exendin-4, a toxin from the Gila Monster), is an FDA-approved therapy for Diabetes Mellitus. This drug is a GLP-1 (glucagon-like-peptide-1) agonist which may act as a survival factor for dopaminergic neurons by functioning as a microglia-deactivating factor (24). It also can reduce inflammation (25), the accumulation of misfolded α-synuclein (26) and improve mitochondrial function (27, 28). Recent preclinical studies suggest that exentatide may be a valuable therapeutic agent for PD. In a recent clinical trial, Exenatide had positive effects on motor scores in Parkinson's disease patients (29).

GLP-1 is a 30- or 31-amino-acid-long peptide hormone deriving from the tissue-specific posttranslational processing of the proglucagon peptide. It is produced and secreted by intestinal enteroendocrine L-cells and certain neurons within the nucleus of the solitary tract in the brainstem upon food consumption. There is a receptor of GLP-1, the glucagon-like peptide-1 receptor (GLP1R). This receptor protein is found on beta cells of the pancreas and on neurons of the brain. It is a member of the glucagon receptor family of G protein-coupled receptors.

Other GLP-1 agonists that may be of value in this invention include:
Dulaglutide (TRULICITY®), taken by injection weekly
Exenatide extended release (BYDUREON BCISE®), taken by injection weekly
Exenatide (BYETTA®), taken by injection twice daily
ORMD-0901-(oral Exenatide taken by mouth once daily)
Semaglutide (OZEMPIC®), taken by injection weekly
Semaglutide (RYBELSUS®), taken by mouth once daily
Liraglutide (VICTOZA®), taken by injection daily
Lixisenatide (ADLYXIN®), taken by injection daily

A proposed dose for the clinical use of exenatide is up to 2 mg subcutaneously once a week.

GCase Inducer

Reduced glucocerebrosidase (GCase) enzymatic activity is found in sporadic cases of Parkinson's disease. GCase deficiency causes accumulation of α-synuclein toxic oligomers in lysosomes which is reported to inhibit mitochondrial protein import in PD. Ambroxol is a secretolytic agent (not approved in the US but used elsewhere) for the treatment of respiratory diseases associated with excessive mucus. Ambroxol is reported to increase GCase activity in different brain-regions which might reduce the accumulation of the misfolded α-synuclein making it a potential therapy for PD (30). In an open label clinical trial an improvement in the clinical motor scores was suggested with the drug (31).

Other GCase enhancers that may be of value in this invention include: BIA 28-5156 (LTI-291); isofagomine: LB-205 and S-181.

A proposed dose for the clinical use of ambroxol is up 420 mg orally 3 times per day.

Iron Chelators

Deferiprone is an FDA-approved therapy for systemic iron (Fe(II)) overload that can cross the blood brain barrier and chelate excessive brain iron from the substantia nigra where it is believed to over accumulate and cause Reactive Oxygen Species (ROS) production and oxidative stress on dopaminergic neurons and dopamine in Parkinson's Disease patients. DFP has the advantage among other iron chelators for its ability to cross membranes, including the blood brain barrier,(32) and to chelate components of the cellular labile iron pool in brain tissue.(33)

In a small double-blind placebo controlled clinical trial in PD patients, Deferiprone showed improvement in both substantia nigra iron deposits (as seen on MRI) and motor scores of disease progression (33).

Other Iron chelators include:
Deferoxamine (DFO), desferrioxamine, deferasirox, clioquinol, tetrahydrosalen, 5,7-Dichloro-2-[(dimethylamino)methyl]quinolin-8-ol (PBT2), (N,N,N,N-Tetrakis (2-pyridylmethyl)-ethylenedi-amine) (TPEN), 1,10-phenanthroline (PHEN), 1,2-hydroxypyridinone (1,2-HOPO).
8-Hydroxyquinolines analogs/derivatives such as clioquinol; 5-[N-methyl-N-propargylaminomethyl]-8-hydroxyquinoline dihydrochloride (M30); M31; M32; 5-[4-(2-hydroxyethyl) piperazine-1-ylmethyl]-quinoline-8-ol (VK28) (Formula 1);
M10 (containing a peptide NAPVSIPQ and an iron-chelating moiety) is a multifunctional 8-hydroxyquinoline which contains a bound alanine residue and exploits the amino acid transporter system that carries L-dopa into the brain.
Prochelators such as SIH-B and BSIH (derived from salicylaldehyde isocotinoyl hydrazone which is then converted to the active non-specific iron chelator SIH during oxidative stress),
Arylhydrazones: pyridoxal isonicotinoyl hydrazine (PIH); 2-pyridylcarboxaldehyde isonicotinoyl hydrazone (PCIH). To increase the activity of PIH series of compounds, several neutral diacylhydrazones were synthesized (C-D), which can get oxidized into hydrazone, thereby forming a neutral complex capable of crossing mitochondrial membrane (Formula 2).

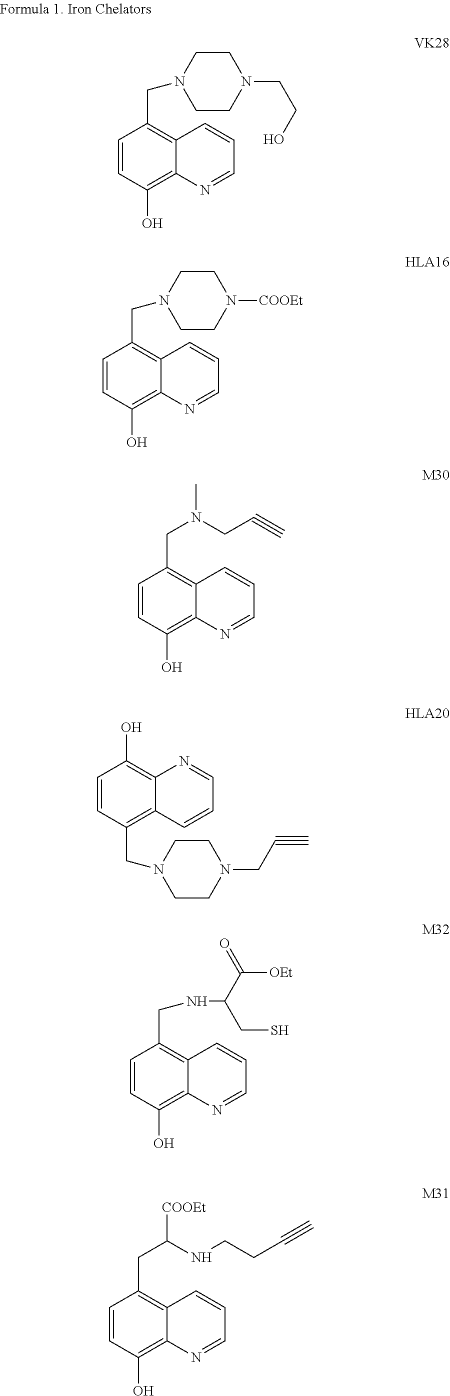 [SMILES: OCCN1CCN(Cc2ccc(O)c3ncccc23)CC1 ; CCOC(=O)N1CCN(Cc2ccc(O)c3ncccc23)CC1 ; C#CCN(C)Cc1ccc(O)c2ncccc12 ; C#CCN1CCN(Cc2ccc(O)c3ncccc23)CC1 ; CCOC(=O)C(CS)NCc1ccc(O)c2ncccc12 ; C#CCCNC(Cc1ccc(O)c2ncccc12)C(=O)OCC]

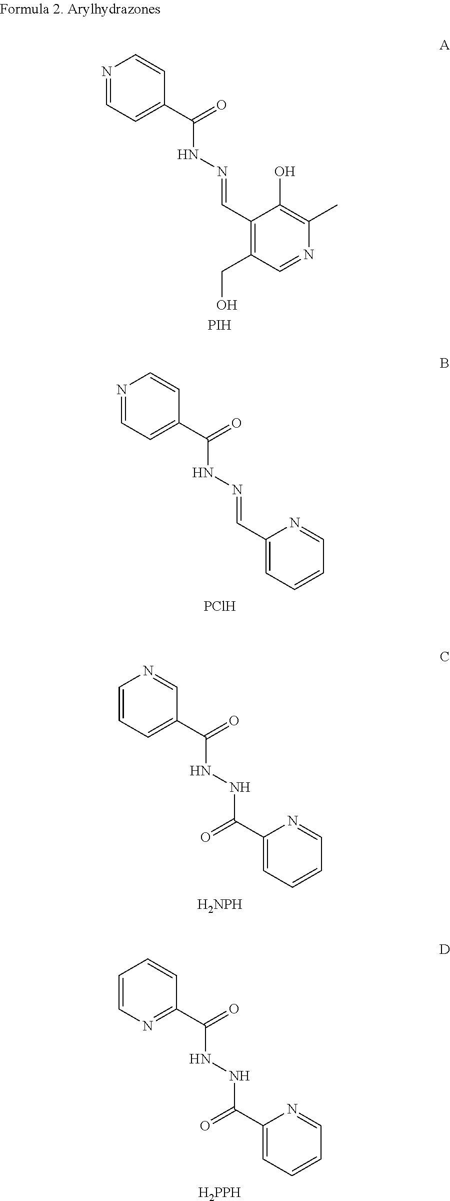

A proposed dose for the clinical use of deferiprone is up to 30 mg/kg/day orally divided in 2 doses.

Mitochondrial Enhancers

Creatine is a guanidine compound, which plays a key role in energy buffering within the cell, which is important in tissues with high and fluctuating energy demands such as brain and muscle (34). A randomized, double-blind, futility clinical trial of creatine in PD concluded it should be studied further (35). A proposed dose for the clinical use of creatine is up to 10 g daily divided in 2 doses.

Coenzyme Q10 (CoQ10), a natural supplement, regulates ATP production and reduces free radical generation. CoQ10 also serves as an important antioxidant in mitochondria and is classified as a mitochondrial enhancer. Effects of CoQ10 in PD patients have been studied extensively and results are mixed, but studies suggest CoQ10 may slow functional decline in early stages of the disease (36). A proposed dose for the clinical use of CoQ10 is up to 1200 mg/day divided in 2 doses.

Although both CoQ10 and creatine have effects on bioenergetics, they act on different pathways. Both CoQ10 and creatine exert neuroprotective effects both in vitro and in vivo in animal models of neurodegenerative diseases (37). The combination of the two agents produced additive neuroprotective effects against dopamine depletion in the striatum in mouse models of PD. The combination treatment resulted in significant reduction in pathologic α-synuclein accumulation in the substantia nigra neurons in the 1-methyl-4-phenyl-1,2,3,6 tetrahydropyridine (MPTP) mouse model of PD (38). Therefore, these two compounds may be used together.

C-Abl Tyrosine Kinase Inhibitors

Nilotinib is FDA approved drug for treating chronic myeloid leukemia (CML). Nilotinib functions as an inhibitor of cluster-Abelson (c-Abl) tyrosine kinase leading to strong autophagy induction. Activity of this kinase is involved either directly or indirectly in increasing α-synuclein levels, intracellular proteins whose toxic misfolded forms are strongly implicated in the pathogenesis of PD. Administration of low-dose nilotinib penetrates the blood-brain barrier and has been shown to reduce inflammation, inhibits brain c-Abl and enhance autophagic clearance of intraneuronal α-synuclein in A53T transgenic mice and lentiviral gene transfer models of PD (39). c-Abl may also be a therapeutic target to mitigate prion-mediated neurotoxicity. In Phase 2 clinical trials for PD, Nilotinib was well tolerated and resulted in favorable changes in exploratory biomarkers of PD pathophysiology (40),(41).

Other c-Abl inhibitors that may be of value in this invention include: radotinib, vodobatinib (K0706), bafetinib, imatinib, dasatinib, bosutinib, ponatinib, rebastinib, tozasertib, and danusertib.

A proposed dose for the clinical use of Nilotinib is up to 300 MG daily divided in 2 doses.

We believe that various combinations of the above agents targeting different disease mechanisms simultaneously may show improved synergistic efficacy in slowing down Parkinson's Disease, DLBD and MSA progression and allow agents to be utilized at lower doses to minimize side effects. In the present study, we therefore, demonstrated that some of the above listed medications used in combination can exert additive neuroprotective effects in preclinical PD models superior to each of the compounds individually. These results can be extrapolated to all α-synucleinopathies such as Parkinson's Disease, DLBD and MSA.

In-Vitro Studies

Neuroprotective Effect of Drugs Against MPP+-Induced Dopaminergic Neurodegeneration

We conducted an in vitro study of the drugs in Table 1 to evaluate the neuroprotective effects of 44 conditions of treatment (Table 2, experiments 1-41; 51-53) against MPP+-induced neurodegeneration toxicity on human dopaminergic neurons (iCell® DopaNeurons: iPS cell-derived human midbrain floorplate dopaminergic neurons). Further combinations (Table 2, experiments 42-50 and Table 3) are also disclosed.

MPP+ is 1-methyl-4-phenylpyridinium, a derivative of MPTP and a known neurotoxin which acts by interfering with oxidative phosphorylation in mitochondria by inhibiting complex I, a protein in the membrane of mitochondria in dopaminergic neurons in the substantia nigra. The inhibition of mitochondrial function leads to the depletion of ATP and eventual cell death. MPP+ ultimately causes Parkinsonism in primates by killing certain dopamine-producing neurons in the substantia nigra. The ability of MPP+ to induce Parkinsonism has made it an important compound in Parkinson's research.

iCell DOPA neurons are neural floor plate-derived midbrain dopaminergic neurons generated from human induced pluripotent stem cells (iPSCs). Dopaminergic neurons, specifically those located in the floor plate-derived midbrain are implicated in neurological disorders such as Parkinson's disease, MSA and DLBD among others. Thus iCell DopaNeurons provide a highly relevant in vitro model to investigate these types of pathologies (42, 43). The iCellDopa neurons were supplied from FUJIFILM Cellular Dynamics.

Each compound in Table 1 was studied in the vehicle solution indicated in the last column.

Three different measurements were made in the experiments. 1. Neurite length in mm is a measure of increase in dendrite length of neurons. Healthy cells exhibited significant increases in neurite length under the control conditions. 2. neurite branching is measure of dendrite branching. Healthy cells exhibited significant increases in neurite branching under the control conditions. 3. Cytolysis of the cells is a measure of cell death. Healthy cells exhibited reduced cytolysis. Experimental conditions were measured relative to the results of healthy cells in the studies.

TABLE 1
Compounds

| Compound | | | | Testing | | |
|---|---|---|---|---|---|---|
| Code | Compound | Provider | Stock | concentration | | Final Vehicle |
| PBA1 | Sodium | TOCRIS | 100 mM in | 500 | μM | 0.5% H2O |
| | Phenylbutyrate | cat#2682 | H2O | | | |
| TZ 2 | Terazosin | TOCRIS | 50 mM in | 10 | μM | 0.02% H2O |
| | | cat#1506 | H2O | | | |
| EXD 3 | Exendin-4 | SIGMA | 200 μM in | 100 | nM | Culture |
| | (Exenatide) | cat#E7144 | culture | | | medium |
| | | | medium | | | |
| AMB 4 | Ambroxol | TOCRIS | 50 mM | 30 | μM ( | 0.06% DMSO |
| | | cat#2404 | DMSO | | | |
| DFP 5 | Deferipone | Sigma Aldich | 50 mM | 50 | μM | 0.1% DMSO |
| | | Cat#Y0001976 | DMSO | | | |
| CR 6 | Creatine | SIGMA | 100 mM in | 10 | μM | Culture |
| | | cat#C0780 | culture | | | medium |
| | | | medium | | | |
| CoQ10 7 | CoQ10 | TOCRIS cat# | 5 mM in | 1 | μM | Ethanol |
| | | 3003 | ethanol | | | |
| NL 8 | Nilotinib | Sigma Cat # | 50 mg/mL in | 300 | nM | 0.06% DMSO |
| | | CDS023093 | DMSO | | | |
| TUDCA 9 | TUDCA | Cat# 580549 | 250 mM | 50 | μM | 100% H2O |
| MPP+ | MPP+ | Sigma D048 | | 100 | μM | Culture |

medium

Control and Vehicle. The culture medium was BrainPhys™ Neuronal Medium, supplied by Stemcell Technologies, www.stemcell.com. Vehicles were culture medium alone, 0.52% H₂O and 0.2% ethanol in culture medium and 0.52% H₂O and 0.16% DMSO in culture medium.

Comparison Substance (positive Control). Pan Caspase inhibitor (PCI) Q-VD-OPh (Bio-Techne, R&D systems, Reference No. OPH001) prepared at 30 μM in 100% DMSO. Q-VD-OPh is effective in preventing apoptosis mediated by three major apoptotic pathways and is used as a positive control for apoptosis research in in vitro and in vivo models (44).

Dosage forms preparation. The solubility of the test substances was checked before each experiment. Each test substance was prepared at the stock solution and working concentration described below to evaluate its solubility. The test substances were prepared freshly on the day of the experiment.

The drugs listed in Table 1 were prepared in stock solutions in the concentrations noted in Table 1. The stock solutions were diluted in the culture medium or other solvents as noted in Table 1.

Procedure. iCell DOPA neurons were thawed and cultured following the provider's instructions in Brainphys medium+provided supplements+1% N2 supplement (Stemcell Technologies)+penicillin/streptomycin+Laminin. They were plated at 20000 cells per well of a 384 well plate (pre-coated with poly-D-lysine and Laminin) in 70 μL of growth medium. Cells were incubated at 37° C./5% CO2 in a humidified cell culture incubator. Half of the culture medium was changed twice a week.

MPP+-induced neurotoxicity: 24 hours after neuronal plating, half of the medium was removed and the test compounds were applied with MPP+ treatment, both concentrated at 4×, were added to the wells. The various combinations and conditions studied are tabulated in Table 2. For each condition, six aliquots were added, corresponding to columns A-F in Table 2. Except for the NT/NT row, in each row, either the compound indicated dissolved in its vehicle noted in Table 1 was added, or only a vehicle was added as indicated. Column F is an indicator of whether MPP+ was added. If not, only the medium was added.

The doses of the drugs used were based on published literature as follows: PBA-500 μM (10); Terazosin-10 μM (22); EXD-100 nM ((45) and Porsolt experience); Ambroxol-30 μM (46); Deferiprone-50 μM (47); Creatine-10 μM (48); CoQ10-1 μM (49); Nilotinib-300 nM (50); TUDCA-50 μM (17).

For the evaluation of conditions 1-28, Neurite outgrowth was followed for subsequent 48 hours using an Incucyte Zoom platform with one phase contrast image every 4 hours, using a 20× objective. After 48 hours, the medium containing test compounds and MPP+ was removed and replaced by fresh medium containing a fluorescent cytolysis marker (red fluorescence) and a live cell marker (green fluorescence). For the evaluation of conditions 29-53, Neurite outgrowth was followed similarly for subsequent 72 hours after MPP+ was added.

TABLE 2
Experimental Combinations
Combinations Studied

| Condition No. | A | B | C | D | E | F |
|---|---|---|---|---|---|---|
| Non-treated | Medium | Medium | Medium | Medium | Medium | Medium |
| NT/NT | | | | | | |
| Control for | 0.5% | 0.02% | Medium | Medium | 0.2% | Medium |
| #1 to #16 | H2O | H2O | | | EtOH | |
| (Control/NT) | | | | | | |
| Control for | 0.5% | 0.02% | Medium | Medium | 0.2% | MPP+ |
| #1 to #16 | H2O | H2O | | | EtOH | |
| (Control/MPP+) | | | | | | |
| #1 | PBA | TZ | EXD | CR | CoQ10 | Medium |
| #2 | PBA | TZ | EXD | CR | CoQ10 | MPP+ |
| #3 | 0.5% | TZ | EXD | CR | CoQ10 | MPP+ |
| | H2O | | | | | |
| #4 | PBA | 0.02% | EXD | CR | CoQ10 | MPP+ |
| | | H2O | | | | |
| #5 | PBA | TZ | Medium | CR | CoQ10 | MPP+ |
| #6 | PBA | TZ | EXD | Medium | 0.2% | MPP+ |
| | | | | | EtOH | |
| #7 | 0.5% | TZ | EXD | Medium | 0.2% | MPP+ |
| | H2O | | | | EtOH | |
| #8 | PBA | 0.02% | EXD | Medium | 0.2% | MPP+ |
| | | H2O | | | EtOH | |
| #9 | PBA | TZ | Medium | Medium | 0.2% | MPP+ |
| | | | | | EtOH | |
| #10 | PBA | 0.02% | Medium | Medium | 0.2% | MPP+ |
| | | H2O | | | EtOH | |
| #11 | 0.5% | TZ | Medium | Medium | 0.2% | MPP+ |
| | H2O | | | | EtOH | |
| #12 | 0.5% | 0.02% | EXD | Medium | 0.2% | MPP+ |
| | H2O | H2O | | | EtOH | |
| #13 | 0.5% | 0.02% | Medium | CR | CoQ10 | MPP+ |
| | H2O | H2O | | | | |
| #14 | PBA | 0.02% | Medium | CR | CoQ10 | MPP+ |
| | | H2O | | | | |
| #15 | 0.5% | TZ | Medium | CR | CoQ10 | MPP+ |
| | H2O | | | | | |
| #16 | 0.5% | 0.02% | EXD | CR | CoQ10 | MPP+ |
| | H2O | H2O | | | | |
| Control for | 0.5% | 0.02% | Medium | 0.06% | 0.1% | Medium |
| #17 to #50 | H2O | H2O | | DMSO | DMSO | |
| Control for | 0.5% | 0.02% | Medium | 0.06% | 0.1% | MPP+ |
| #17 to #50 | H2O | H2O | | DMSO | DMSO | |
| #17 | PBA | TZ | EXD | AMB | 0.1% | MPP+ |
| | | | | | DMSO | |
| #18 | PBA | TZ | Medium | AMB | 0.1% | MPP+ |
| | | | | | DMSO | |
| #19 | 0.5% | 0.02% | Medium | AMB | DFP | MPP+ |
| | H2O | H2O | | | | |
| #20 | PBA | 0.02% | Medium | AMB | 0.1% | MPP+ |
| | | H2O | | | DMSO | |
| #21 | PBA | 0.02% | Medium | AMB | DFP | MPP+ |
| | | H2O | | | | |
| #22 | PBA | 0.02% | Medium | 0.06% | DFP | MPP+ |
| | | H2O | | DMSO | | |
| #23 | 0.5% | 0.02% | Medium | AMB | 0.1% | MPP+ |
| | H2O | H2O | | | DMSO | |
| #24 | 0.5% | 0.02% | Medium | 0.06% | DFP | MPP+ |
| | H2O | H2O | | DMSO | | |
| #25 | PBA | TZ | EXD | AMB | DFP | Medium |
| #26 | PBA | TZ | EXD | AMB | DFP | MPP+ |
| #27 | PBA | TZ | Medium | AMB | DFP | MPP+ |
| #28 | PBA | TZ | Medium | 0.06% | DFP | MPP+ |
| | | | | DMSO | | |
| #29 | 0.5% | TZ | EXD | AMB | DFP | MPP+ |
| | H2O | | | | | |
| #30 | 0.5% | 0.02% | EXD | AMB | DFP | MPP+ |
| | H2O | H2O | | | | |
| #31 | PBA | 0.02% | EXD | AMB | DFP | MPP+ |
| | | H2O | | | | |
| #32 | PBA | TZ | EXD | 0.06% | DFP | MPP+ |
| | | | | DMSO | | |
| #33 | 0.5% | TZ | Medium | AMB | DFP | MPP+ |
| | H2O | | | | | |
| #34 | 0.5% | TZ | EXD | AMB | 0.1% | MPP+ |
| | H2O | | | | DMSO | |
| #35 | 0.5% | TZ | Medium | 0.06% | DFP | MPP+ |
| | H2O | | | DMSO | | |
| #36 | 0.5% | TZ | Medium | AMB | 0.1% | MPP+ |
| | H2O | | | | DMSO | |
| #37 | 0.5% | 0.02% | EXD | AMB | 0.1% | MPP+ |
| | H2O | H2O | | | DMSO | |
| #38 | 0.5% | 0.02% | EXD | 0.06% | DFP | MPP+ |
| | H2O | H2O | | DMSO | | |
| #39 | 0.5% | TZ | EXD | 0.06% | DFP | MPP+ |
| | H2O | | | DMSO | | |
| #40 | PBA | 0.02% | EXD | 0.06% | DFP | MPP+ |
| | | H2O | | DMSO | | |
| #41 | PBA | 0.02% | EXD | AMB | 0.1% | MPP+ |
| | | H2O | | | DMSO | |
| #42 | PBA | TZ | EXD | NL | | Medium |
| #43 | PBA | TZ | EXD | NL | | MPP+ |
| #44 | 0.5% | 0.02% | Medium | NL | | MPP+ |
| | H2O | H2O | | | | |
| #45 | PBA | 0.02% | Medium | NL | | MPP+ |
| | | H2O | | | | |
| #46 | 0.5% | TZ | Medium | NL | | MPP+ |
| | H2O | | | | | |
| #47 | 0.5% | 0.02% | EXD | NL | | MPP+ |
| | H2O | H2O | | | | |
| #48 | PBA | 0.02% | EXD | NL | | MPP+ |
| | | H2O | | | | |
| #49 | 0.5% | TZ | EXD | NL | | MPP+ |
| | H2O | | | | | |
| #50 | PBA | TZ | Medium | NL | | MPP+ |
| #51 | 0.5% | TUDCA | Medium | Medium | 0.2% | MPP+ |
| | H2O | | | | EtOH | |
| #52 | PBA | TUDCA | Medium | Medium | 0.2% | MPP+ |
| | | | | | EtOH | |
| #53 | PBA | TUDCA | EXD | Medium | 0.2% | MPP+ |
| | | | | | EtOH | |
| Positive | PCI | — | — | — | — | MPP+ |
| control | (30 μM) | | | | | |

Table 2 compound key (see also Table 1):
1. Sodium Phenylbutyrate (PBA) (500 μM)
2. Terazosin (TZ) (10 μM)
3. Exendin-4 (Exenatide, EXD) (100 nM)
4. Ambroxol (AMB) (30 μM)
5. Deferiprone (DFP) (50 μM)
6. Creatine (CR) (10 μM)
7. CoQ10 (1 μM)
8. Nilotinib (NL) (300 nM)
9. TUDCA (50 μM)

Each condition 1-41 and 51-53 was repeated in 8 wells of a 384 well plate, that is, each test condition studied in Table 2 was repeated eight times. NT/NT were cells in the standard medium only. Control/NT are the cells in the vehicle (as described above), with and without MPP+.

Table 3 tabulates an expanded list of combinations in summary form with the combinations from Table 2 and additional combinations 54-94.

TABLE 3
Drug Combinations

| Combination | Drugs in combination |
|---|---|
| number | (all combinations have MPP+ unless otherwise noted) |
| 1 | PBA, EXD, CR, CQ, TZ (no MPP+) |
| 2 | PBA, EXD, CR, CQ, TZ |
| 3 | TZ, EXD, CR, CoQ10 |
| 4 | PBA, EXD, CR, CQ |
| 5 | PBA, CR, CQ, TZ |
| 6 | PBA, EXD, TZ |
| 7 | TZ, EXD |
| 8 | PBA, EXD |
| 9 | PBA, TZ |
| 10 | PBA |
| 11 | TZ |
| 12 | EXD |
| 13 | CR, CoQ10 |
| 14 | PBA, CR, CoQ10 |
| 15 | TZ, CR, CoQ10 |
| 16 | EXD, CR, CoQ10 |
| 17 | PBA, TZ, EXD, AMB |
| 18 | PBA, TZ, AMB |
| 19 | AMB, DFP |
| 20 | PBA, AMB |
| 21 | PBA, AMB, DFP |
| 22 | PBA, DFP |
| 23 | AMB |
| 24 | DFP |
| 25 | PBA, TZ, EXD, AMB, DFP (no MPP+) |
| 26 | PBA, TZ, EXD, AMB, DFP |
| 27 | PBA, TZ, AMB, DFP |
| 28 | PBA, TZ, DFP |
| 29 | TZ, EXD, AMB, DFP |
| 30 | EXD, AMB, DFP |
| 31 | PBA, EXD, AMB, DFP |
| 32 | PBA, TZ, EXD, DFP |
| 33 | TZ, AMB, DFP |
| 34 | TZ, EXD, AMB |
| 35 | TZ, DFP |
| 36 | TZ, AMB |
| 37 | EXD, AMB |
| 38 | EXD, DFP |
| 39 | TZ, EXD, DFP |
| 40 | PBA, EXD, DFP |
| 41 | PBA, EXD, AMB |
| 42 | PBA, EXD, NL, TZ (no MPP+) |
| 43 | PBA, EXD, NL, TZ |
| 44 | NL |
| 45 | PBA, NL |
| 46 | TZ, NL |
| 47 | EXD, NL |
| 48 | PBA, EXD, NL |
| 49 | EXD, NL, TZ |
| 50 | PBA, TZ, NL |
| 51 | TUDCA |
| 52 | PBA, TUDCA |
| 53 | PBA, EXD, TUDCA |
| 54 | NL, TUDCA, TZ |
| 55 | EXD, NL, TUDCA, TZ |
| 56 | CR, CQ, NL, TUDCA, TZ |
| 57 | PBA, CR, CQ, TUDCA |
| 58 | EXD, NL, TUDCA |
| 59 | PBA, CR, CQ, TUDCA, TZ |
| 60 | EXD, CR, CQ, NL, TZ |
| 61 | EXD, NL, TUDCA, TZ |
| 62 | PBA, TUDCA, TZ |
| 63 | PBA, EXD, TUDCA, TZ |
| 64 | EXD, TUDCA, TZ |
| 65 | EXD, CR, CQ, TUDCA, TZ |
| 66 | PBA, NL, TUDCA, TZ |
| 67 | CR, CoQ10, NL |
| 68 | PBA, EXD, NL, TUDCA, TZ |
| 69 | PBA, EXD, NL, DFP |
| 70 | PBA, CR, CQ, DFP |
| 71 | PBA, CR, CQ, NL, DFP |
| 72 | PBA, CR, CQ, TUDCA, DFP |
| 73 | NL, TUDCA, DFP |
| 74 | PBA, TUDCA, DFP |
| 75 | EXD, NL, TUDCA, DFP |
| 76 | CR, CQ, NL, TUDCA, DFP |
| 77 | PBA, EXD, CR, CQ, DFP |
| 78 | EXD, NL, DFP |
| 79 | EXD, CR, CQ, NL, DFP |
| 80 | EXD, NL, TUDCA, DFP |
| 81 | EXD, TUDCA, DFP |
| 82 | EXD, CR, CQ, TUDCA, DFP |
| 83 | PBA, NL, TUDCA, DFP |
| 84 | PBA, EXD, NL, TUDCA, DFP |
| 85 | PBA, EXD, TUDCA, DFP |
| 86 | NL, TUDCA |
| 87 | PBA, NL, TUDCA |
| 88 | PBA, CR, CQ, NL, TZ |
| 89 | EXD, TUDCA |
| 90 | EXD, CR, CQ, TUDCA |
| 91 | CR, CQ, NL, TUDCA |
| 92 | EXD, CR, CQ, NL |
| 93 | PBA, CR, CQ, NL |
| 94 | PBA, EXD, NL, TUDCA |

Legend:
PBA—Sodium Phenylbutyrate
TZ—Terazosin
EXD—Exendin-4 (Exenatide)
AMB—Ambroxol
DFP—Deferiprone
CR—Creatine
CoQ10
NL—Nilotinib (not studied in this table)
TUDCA—Tauroursodeoxycholic acid
MPP+—1-methyl-4-phenylpyridinium
PCI—Pan Caspase inhibitor (Positive Control)

Assay endpoints and data analysis. Phase contrast images were analyzed at each time point to determine the neurite length and number of branch points per mm². Kinetic data was plotted and kinetics were normalized by subtracting the value of the first data point (at time of treatment), allowing to measure changes in neurite outgrowth only from the onset of the treatment, starting at zero. Area Under Curve (AUC) of kinetic data was obtained and used for plotting compound's effects and perform statistical analyzes.

Data was normalized to control conditions as there is basal cytolysis in the cell culture after thawing.

Fluorescently immuno-stained cells were imaged on a high content imaging platform. Individual segmentation of cells was performed by image analysis of the MAP2 and NeuN staining. The number of neurons and the neurite length were measured. The percentage of cytolyzed cells was also calculated.

Statistical analysis. We used one-way ANOVA followed by Bonferroni's multiple comparisons test.

Results for MPP+-Induced Neurotoxicity on ICellDopa Neurons

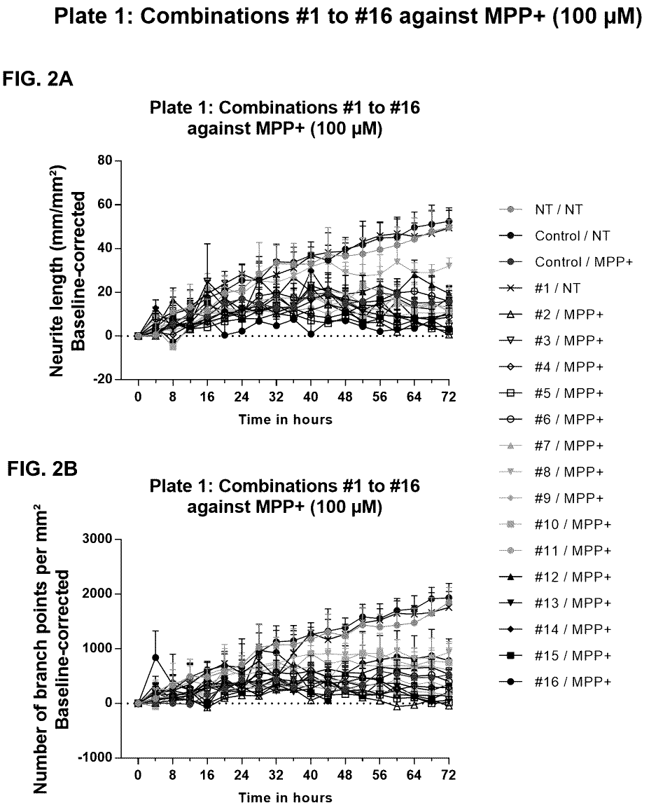

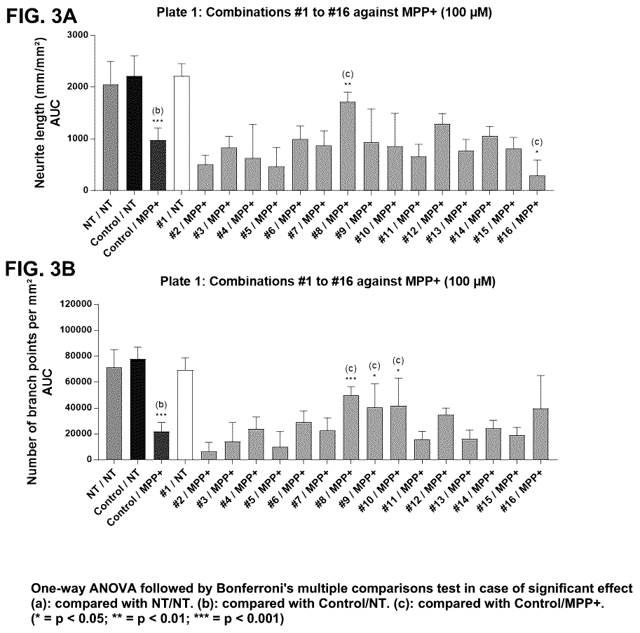

Plate 1, FIGS. 2A, 2B, 3A, 3B

In vehicle conditions, neurite outgrowth, as compared with Non-Treated (NT) conditions. MPP+ at 100 μM induced a high inhibition of neurite outgrowth (AUC of the neurite length and the number of branch), as compared with NT conditions (p≤0.001 for both conditions).

In Condition #1 (Phenylbutyrate, Terazosin, exenatide, Creatine and CoQ10), the neurite outgrowth was not affected as compared with NT conditions.

Condition #8 (Phenylbutyrate and exenatide), show synergistic and promising results. The neurite length AND the number of branches was 75% increased as compared with MPP+ alone (p≤0.05 and p≤0.001, respectively). The drugs tested individually (samples 10 and 12) showed the protective effect, but to a substantially lesser effect than when combined.

In conditions #9 (Phenylbutyrate and Terazosin) and 10 (Phenylbutyrate), the number of branches was significantly increased, as compared with MPP+ alone (p≤0.05 for each condition).

In conditions #16 exenatide, creatine and CoQ10), the neurite length was slightly but significantly decreased, as compared with MPP+ alone (p≤0.05).

In conditions #4, 6, 7, 11 and 12, no significant effects were observed on neurite outgrowth.

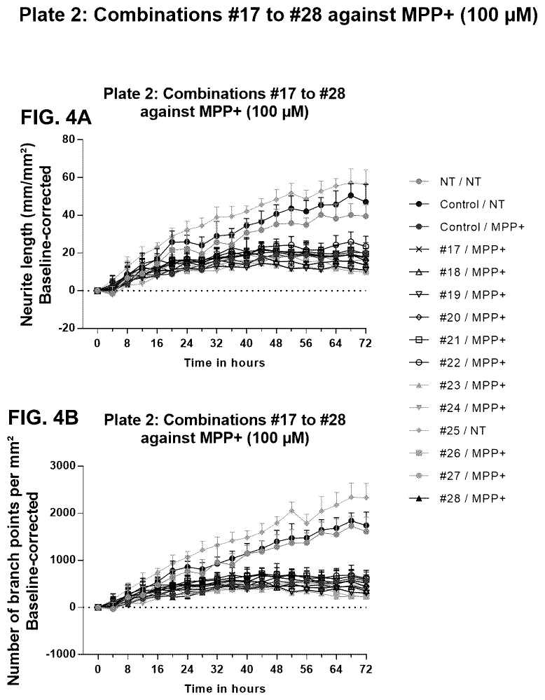

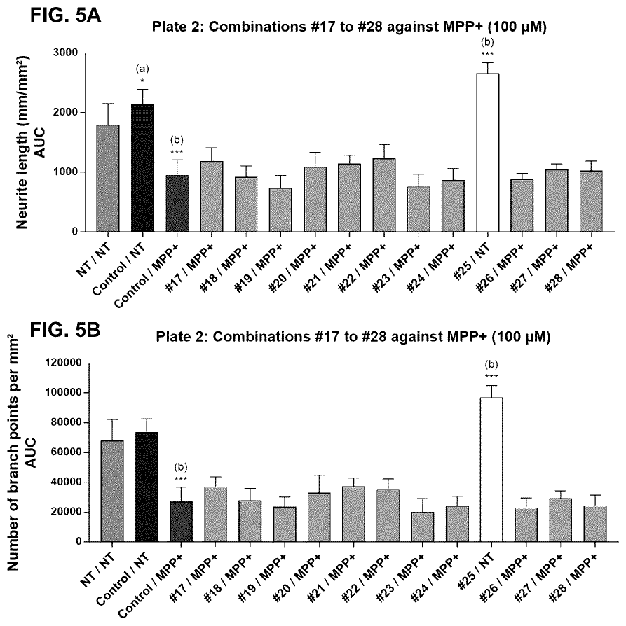

Plate 2 FIGS. 4A, 4B, 5A, 5B

In vehicle conditions, neurite length was slightly but significantly increased, as compared with NT conditions (p≤0.05).

MPP+ at 100 μM induced a high inhibition of neurite outgrowth (AUC of the neurite length and the number of branch), as compared with Non-Treated (NT) conditions (p≤0.001 for both conditions).

Condition #25 (Phenylbutyrate, Terazosin, Exenatide, Ambroxol and Deferiprone), significantly increased the neurite length and the number of branch, as compared with NT conditions (p≤0.001 for both conditions).

In conditions #17, 26 and 28, no significant effects were observed on neurite outgrowth for these conditions.

In conditions #18, 19, 20, 21, 22, 23 and 24, no significant effects were observed on neurite outgrowth.

Results for the combinations tested are reported in Tables 4-xx below. Each condition was replicated eight times and the mean is reported.

TABLE 4.1
Neurite length data. (Plotted in FIG. 3A).
Neurite length (mm/mm²) - Baseline-corrected
AUC (0-72 h)

| | | | | Stat vs | Stat vs | Stat vs |
|---|---|---|---|---|---|---|
| Treatment | Mean | ± | S.D. | NT/NT | Control/NT | Control/MPP+ |
| NT/NT | 2057.5 | ± | 440.3 | | | |
| Control/NT | 2215.6 | ± | 387.4 | NS | | |
| Control/MPP+ | 977.2 | ± | 230.6 | | *** | |
| #1/NT | 2215.3 | ± | 236.4 | | NS | |
| #2/MPP+ | 511.4 | ± | 173 | | | NS |
| #3/MPP+ | 837.1 | ± | 214.8 | | | NS |
| #4/MPP+ | 631.9 | ± | 652.5 | | | NS |
| #5/MPP+ | 468.8 | ± | 363.7 | | | NS |
| #6/MPP+ | 998 | ± | 253.4 | | | NS |
| #7/MPP+ | 879.6 | ± | 277.2 | | | NS |
| #8/MPP+ | 1718.7 | ± | 187.9 | | | ** |
| #9/MPP+ | 930.7 | ± | 647.3 | | | NS |
| #10/MPP+ | 853.7 | ± | 643.8 | | | NS |
| #11/MPP+ | 661 | ± | 231.8 | | | NS |
| #12/MPP+ | 1294.5 | ± | 196.2 | | | NS |
| #13/MPP+ | 778.9 | ± | 210.7 | | | NS |
| #14/MPP+ | 1054.5 | ± | 186.8 | | | NS |
| #15/MPP+ | 819.1 | ± | 210.7 | | | NS |
| #16/MPP+ | 291.1 | ± | 303.2 | | | * |

NS = Not Significant;
* = p < 0.05;
** = p < 0.01;
*** = p < 0.001.
One-way ANOVA followed by Bonferroni's multiple comparisons test in case of
significant effect.

Based on these results for neurite length, experiment 8 is 75% better than control/MPP+ and exenatide alone (#12) is 32% better than control.

TABLE 4.2
Neurite Branching Data (Plotted in FIG. 3B)
Number of branch points per mm² - Baseline-corrected
AUC (0-72 h)

| | | | Stat vs | Stat vs | Stat vs |
|---|---|---|---|---|---|
| | | | NT/ | Control/ | Control/ |
| Treatment | Mean | ±S.D. | NT | NT | MPP+ |
| NT/NT | 71664.9 | ±13468.8 | | | |
| Control/NT | 78045 | ±9151.5 | NS | | |
| Control/MPP+ | 22082 | ±6853.7 | | *** | |
| #1/NT | 69559.5 | ±9161.3 | | NS | |
| #2/MPP+ | 6334.6 | ±7127.6 | | | NS |
| #3/MPP+ | 14072.1 | ±14782.8 | | | NS |
| #4/MPP+ | 23940.4 | ±9248.6 | | | NS |
| #5/MPP+ | 9976.4 | ±12016.8 | | | NS |
| #6/MPP+ | 29024.8 | ±8723.4 | | | NS |
| #7/MPP+ | 22602 | ±9700 | | | NS |
| #8/MPP+ | 49743.7 | ±6618.8 | | | *** |
| #9/MPP+ | 40677.5 | ±18111.4 | | | * |
| #10/MPP+ | 41712.8 | ±21469.3 | | | * |
| #11/MPP+ | 15987.1 | ±6020.6 | | | NS |
| #12/MPP+ | 34983.1 | ±5097.8 | | | NS |
| #13/MPP+ | 16179 | ±6876.8 | | | NS |
| #14/MPP+ | 24392 | ±6124.9 | | | NS |
| #15/MPP+ | 18869 | ±6224.4 | | | NS |
| #16/MPP+ | 39634 | ±25425.3 | | | NS |

NS = Not Significant;
* = p < 0.05;
** = p < 0.01;
*** = p < 0.001.
One-way ANOVA followed by Bonferroni's multiple comparisons test in case of significant effect.

For the number of branch points tabulated in Table 4.2, experiment 8 was 125% better than control/MPP+, phenylbutyrate alone (#10) is 89% better than control and exenatide (#12) is 58% better than control.

Results for experiments 17-28 are in the attached appendices. The attached appendices include all data from the in vitro experiments 1-28 tabulated in Table 2.

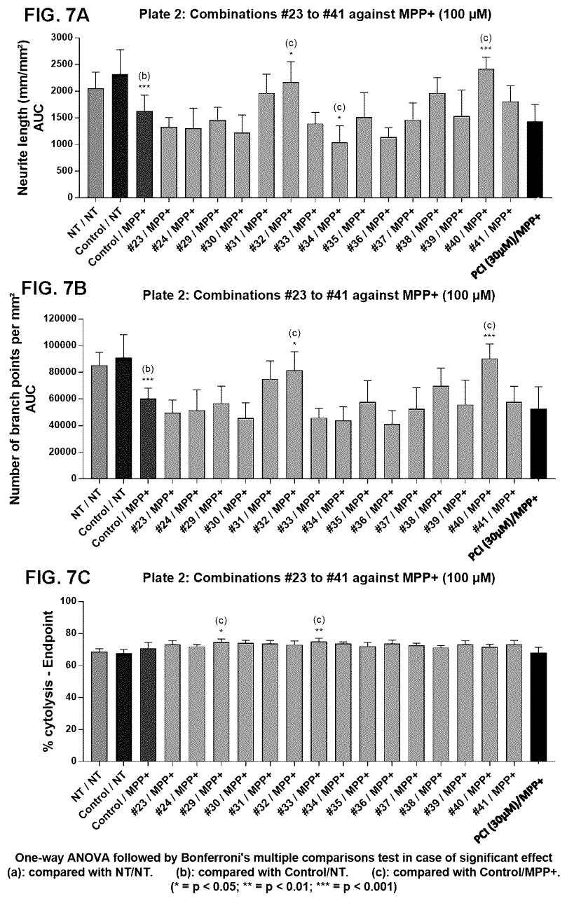

We also identified a combination of sodium phenylbutyrate, exenatide and deferiprone which showed additive protective effect (FIG. 7A, B, sample #40). The neurite length and the number of branches were increased by 48.9% and 49.7% respectively, as compared with MPP+ alone (p≤0.001 for both). Sodium phenylbutyrate, exenatide re-tested individually (samples 10, 12) however, also showed protective effect, but it was not statistically significant.

In condition #32 (PBA, TZ, EXD, DFP), the neurite length and the number of branches were significantly increased, as compared with MPP+ alone (p≤0.05 for each parameter).

In condition #8 (Phenylbutyrate and exenatide), the neurite length AND the number of branches was 75% increased, as compared with MPP+ alone (p≤0.05 and p≤0.001, respectively). However, the drugs tested individually (samples 10 and 12) showed the protective effect, but lesser than when combined (FIG. 3A, 3B) suggesting unexpected enhanced combinatory effect.

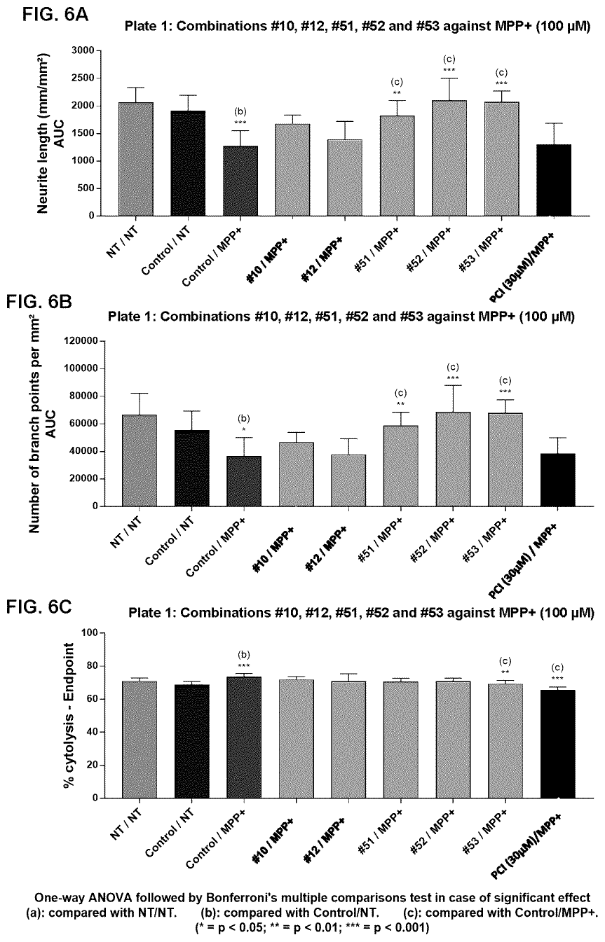

We also found that combinations of TUDCA and Phenylbutyrate (sample 52), as well as TUDCA, Phenylbutyrate and Exenatide (sample 53) increased the neurite length (65.2% and 63.2%) and the number of branches (86.6% and 85.1% respectively), as compared with MPP+-treated cells without the drugs (FIG. 6A, B p≤0.001 for both conditions). The latter combination (#53) also unexpectedly reduced cytolysis of the cells which was statistically significant and was not seen with any other combinations (FIG. 6C). While TUDCA alone (sample 51) protected against MPP+ toxicity as well (43% and 60% as per above respectively), the effect was at least 20% greater when the drugs were combined in conditions 52 or 53. All the tested medications as well as with nilotinib or dasatinib (not yet tested) together are thought to affect most of the known pathways such as aggregation of misfolded proteins, neuroinflammation, mitochondrial dysfunction and iron accumulation leading to neurodegeneration in PD (FIG. 1).

Results for combinations 17-28 are reported in Tables 5.1 and 5.2.

TABLE 5.1
Neurite Length Data (plotted in FIG. 5A)

| | | Stat vs | Stat vs | Stat vs |
|---|---|---|---|---|
| Treatment | Mean ± S.D. | NT/NT | Control/NT | Control/MPP+ |
| NT/NT | 1798.5 ± 355.9 | | | |
| Control/NT | 2148.9 ± 242.4 | * | | |
| Control/MPP+ | 955.3 ± 254.5 | | *** | |
| #17/MPP+ | 1189.1 ± 223.9 | | | NS |
| #18/MPP+ | 925.7 ± 184 | | | NS |
| #19/MPP+ | 744.7 ± 201.4 | | | NS |
| #20/MPP+ | 1090.6 ± 245.5 | | | NS |
| #21/MPP+ | 1148.9 ± 142.8 | | | NS |
| #22/MPP+ | 1234.9 ± 233.5 | | | NS |
| #23/MPP+ | 763.9 ± 209.8 | | | NS |
| #24/MPP+ | 874.5 ± 190.4 | | | NS |
| #25/NT | 2659.6 ± 180.9 | | *** | |
| #26/MPP+ | 890.9 ± 91.7 | | | NS |
| #27/MPP+ | 1046.6 ± 93.8 | | | NS |
| #28/MPP+ | 1028.4 ± 162.3 | | | NS |

TABLE 5.2
Neurite Branching Data (Plotted in FIG. 5B)

| | | Stat vs | Stat vs | Stat vs |
|---|---|---|---|---|
| Treatment | Mean ± S.D. | NT/NT | Control/NT | Control/MPP+ |
| NT/NT | 67918.3 ± 14220.8 | | | |
| Control/NT | 73864 ± 8671.5 | NS | | |
| Control/MPP+ | 27222.8 ± 9644.1 | | *** | |
| #17/MPP+ | 37270.6 ± 6433.1 | | | NS |
| #18/MPP+ | 27978.5 ± 7972.6 | | | NS |
| #19/MPP+ | 23638.3 ± 6622.5 | | | NS |
| #20/MPP+ | 33064.8 ± 11867.2 | | | NS |
| #21/MPP+ | 37458.9 ± 5484.1 | | | NS |
| #22/MPP+ | 35112.5 ± 7198.1 | | | NS |
| #23/MPP+ | 20281.6 ± 8723.4 | | | NS |
| #24/MPP+ | 24282.5 ± 6612.9 | | | NS |
| #25/NT | 97093.8 ± 7854.9 | | *** | |
| #26/MPP+ | 23189 ± 6325.9 | | | NS |
| #27/MPP+ | 29339.9 ± 5025.3 | | | NS |
| #28/MPP+ | 24569.9 ± 6868.4 | | | NS |

Results for the combinations 29-41 and 51-53 are reported in Tables 6, 7 and 8.

TABLE 6.1
Neurite length data, plate 1 (Plotted in FIG. 6A)

| | | | Stat vs | Stat vs | Stat vs |
|---|---|---|---|---|---|
| Treatment | Mean | ±S.D. | NT/NT | Control/NT | Control/MPP+ |
| NT/NT | 2067.3 | ±266.8 | | | |
| Control/NT | 1920.4 | ±275.8 | NS | | |
| Control/MPP+ | 1272.9 | ±280.0 | | *** | |
| #10/MPP+ | 1681.1 | ±156.1 | | | * |
| #11/MPP+ | 1102.2 | ±298.7 | | | NS |
| #12/MPP+ | 1395.3 | ±326.1 | | | NS |
| #51/MPP+ | 1825.9 | ±273.8 | | | *** |
| #52/MPP+ | 2103.1 | ±399.7 | | | *** |
| #53/MPP+ | 2077.3 | ±195.9 | | | *** |
| PCI/MPP+ | 1301.0 | ±387.8 | | | NS |

NS = Not Significant;
* = p < 0.05;
** = p < 0.01;
*** = p < 0.001.
One-way ANOVA followed by Bonferroni's multiple comparisons test in case of significant effect.

TABLE 6.2
Neurite length, plate 2 (Plotted in FIG. 7A)

| | | Stat vs | Stat vs | Stat vs |
|---|---|---|---|---|
| Treatment | Mean ± S.D. | NT/NT | Control/NT | Control/MPP+ |
| NT/NT | 2053.5 ± 304.1 | | | |
| Control/NT | 2323.6 ± 458.5 | NS | | |
| Control/MPP+ | 1623.9 ± 301.2 | | *** | |
| #23/MPP+ | 1332.1 ± 174.8 | | | NS |
| #24/MPP+ | 1303.0 ± 378.7 | | | NS |
| #29/MPP+ | 1462.4 ± 237.3 | | | NS |
| #30/MPP+ | 1221.0 ± 332.7 | | | NS |
| #31/MPP+ | 1967.6 ± 354.6 | | | NS |
| #32/MPP+ | 2174.4 ± 379.8 | | | * |
| #33/MPP+ | 1391.8 ± 212.4 | | | NS |
| #34/MPP+ | 1044.5 ± 307.4 | | | * |
| #35/MPP+ | 1514.0 ± 457.4 | | | NS |
| #36/MPP+ | 1138.8 ± 175.9 | | | NS |
| #37/MPP+ | 1466.9 ± 313.9 | | | NS |
| #38/MPP+ | 1968.3 ± 289.0 | | | NS |
| #39/MPP+ | 1533.6 ± 490.7 | | | NS |
| #40/MPP+ | 2418.3 ± 222.1 | | | *** |
| #41/MPP+ | 1811.9 ± 288.2 | | | NS |
| PCI/MPP+ | 1430.1 ± 321.7 | | | NS |

NS = Not Significant;
* = p < 0.05;
** = p < 0.01;
*** = p < 0.001.
One-way ANOVA followed by Bonferroni's multiple comparisons test in case of significant effect.

TABLE 7.1
Neurite Branching Data, Plate 1 (Plotted in FIG. 6B)

| | | Stat vs | Stat vs | Stat vs |
|---|---|---|---|---|
| Treatment | Mean ± S.D. | NT/NT | Control/NT | Control/MPP+ |
| NT/NT | 66758.3 ± 15442.2 | | | |
| Control/NT | 55661.3 ± 13676.2 | NS | | |
| Control/MPP+ | 36771.0 ± 13342.2 | | * | |
| #10/MPP+ | 46742.8 ± 7107.5 | | | NS |
| #11/MPP+ | 31244.4 ± 12478.9 | | | NS |
| #12/MPP+ | 37977.9 ± 11294.6 | | | NS |
| #51/MPP+ | 58836.3 ± 9596.4 | | | ** |
| #52/MPP+ | 68626.9 ± 19348.6 | | | *** |
| #53/MPP+ | 68062.9 ± 9445.6 | | | *** |
| PCI/MPP+ | 38456.3 ± 11570.7 | | | NS |

NS = Not Significant;
* = p < 0.05;
** = p < 0.01;
*** = p < 0.001.
One-way ANOVA followed by Bonferroni's multiple comparisons test in case of significant effect.

TABLE 7.2
Neurite Branching Data, Plate 2. (Plotted in FIG. 7B).

| | | Stat vs | Stat vs | Stat vs |
|---|---|---|---|---|
| Treatment | Mean ± S.D. | NT/NT | Control/NT | Control/MPP+ |
| NT/NT | 85402.0 ± 9687.5 | | | |
| Control/NT | 91022.9 ± 17191.7 | NS | | |
| Control/MPP+ | 60484.3 ± 7623.4 | | *** | |
| #23/MPP+ | 49875.9 ± 9356.8 | | | NS |
| #24/MPP+ | 51711.3 ± 15178.4 | | | NS |
| #29/MPP+ | 56978.4 ± 12679.8 | | | NS |
| #30/MPP+ | 45806.1 ± 11443.1 | | | NS |
| #31/MPP+ | 75119.8 ± 13385.8 | | | NS |
| #32/MPP+ | 81697.3 ± 13858.5 | | | * |
| #33/MPP+ | 45965.5 ± 6893.3 | | | NS |
| #34/MPP+ | 43947.0 ± 10192.3 | | | NS |
| #35/MPP+ | 57812.9 ± 15983.4 | | | NS |
| #36/MPP+ | 41427.1 ± 9933.0 | | | NS |
| #37/MPP+ | 52764.0 ± 15725.2 | | | NS |
| #38/MPP+ | 69718.3 ± 13499.6 | | | NS |
| #39/MPP+ | 55873.0 ± 18240.9 | | | NS |
| #40/MPP+ | 90598.0 ± 10606.3 | | | *** |
| #41/MPP+ | 57833.5 ± 11708.4 | | | NS |
| PCI/MPP+ | 52610.1 ± 16660.1 | | | NS |

NS = Not Significant;
* = p < 0.05;
** = p < 0.01;
*** = p < 0.001.
One-way ANOVA followed by Bonferroni's multiple comparisons test in case of significant effect.

TABLE 8.1
Cytolysis, %, Plate 1. (Plotted in FIG. 6C)

| | | Stat vs | Stat vs | Stat vs |
|---|---|---|---|---|
| Treatment | Mean ± S.D. | NT/NT | Control/NT | Control/MPP+ |
| NT/NT | 71.10 ± 1.76 | | | |
| Control/NT | 68.82 ± 1.95 | NS | | |
| Control/MPP+ | 73.74 ± 1.85 | | *** | |
| #10/MPP+ | 72.06 ± 1.77 | | | NS |
| #11/MPP+ | 72.78 ± 2.29 | | | NS |
| #12/MPP+ | 70.99 ± 4.42 | | | NS |
| #51/MPP+ | 70.69 ± 2.02 | | | NS |
| #52/MPP+ | 70.89 ± 1.94 | | | NS |
| #53/MPP+ | 69.31 ± 2.15 | | | ** |
| PCI/MPP+ | 65.56 ± 1.89 | | | *** |

NS = Not Significant;
* = p < 0.05;
** = p < 0.01;
*** = p < 0.001.
One-way ANOVA followed by Bonferroni's multiple comparisons test in case of significant effect.

TABLE 8.2
Cytolysis, %, Plate 2. (Plotted in FIG. 7C)

| | | Stat vs | Stat vs | Stat vs |
|---|---|---|---|---|
| Treatment | Mean ± S.D. | NT/NT | Control/NT | Control/MPP+ |
| NT/NT | 68.64 ± 1.97 | | | |
| Control/NT | 67.78 ± 2.31 | NS | | |
| Control/MPP+ | 71.00 ± 3.43 | | NS | |
| #23/MPP+ | 73.29 ± 2.23 | | | NS |
| #24/MPP+ | 71.91 ± 1.28 | | | NS |
| #29/MPP+ | 74.68 ± 1.99 | | | * |
| #30/MPP+ | 74.08 ± 1.77 | | | NS |
| #31/MPP+ | 73.81 ± 1.89 | | | NS |
| #32/MPP+ | 73.12 ± 2.33 | | | NS |
| #33/MPP+ | 75.03 ± 2.11 | | | ** |
| #34/MPP+ | 73.87 ± 0.94 | | | NS |
| #35/MPP+ | 72.13 ± 2.30 | | | NS |
| #36/MPP+ | 73.81 ± 2.23 | | | NS |
| #37/MPP+ | 72.53 ± 1.48 | | | NS |
| #38/MPP+ | 71.18 ± 1.37 | | | NS |
| #39/MPP+ | 73.30 ± 2.21 | | | NS |
| #40/MPP+ | 71.81 ± 1.68 | | | NS |
| #41/MPP+ | 73.31 ± 2.37 | | | NS |
| PCI/MPP+ | 67.93 ± 3.52 | | | NS |

NS = Not Significant;
* = p < 0.05;
** = p < 0.01;
*** = p < 0.001.
One-way ANOVA followed by Bonferroni's multiple comparisons test in case of significant effect.

Experiment 25 shows that in the absence of MPP+, the combination of Sodium Phenylbutyrate, Terazosin, Exenatide, Ambroxol, and Deferiprone showed significantly improved neuronal growth as compared to controls. This suggests that those conditions may have a nurturing effect on dopaminergic neurons and may be effective at preventing the onset of α-synucleinopathies.

Evaluation of Effect of the Drug Combinations on Neuroinflammation Using Human Microglia Cell Line

In addition to the studies discussed above assessing activity of combinations of drugs on MPP+-induced neurodegeneration toxicity on human dopaminergic neurons and measuring neuronal growth and neurite branching, we also assessed combinations of drugs in reducing lipopolysaccharide (LPS, from E. coli)-induced neuroinflammation. Accordingly, a study was conducted to evaluate the anti-neuroinflammatory effects of sodium phenylbutyrate (PBA), Tauroursodeoxycholic acid (TUDCA) and Exendin (EXD) in different combinations by measuring their effect on pro-inflammatory cytokines secretion following LPS and ATP stimulation of human brain microglia cells. As noted in FIG. 1, neuroinflammation is a key pathology in synucleinopathies, so success in reducing neuroinflammation would demonstrate the value of this invention.

Lipopolysaccharides are important outer membrane components of gram-negative bacteria such as E. coli and salmonella. They are large amphipathic glycoconjugates that typically consist of a lipid domain (hydrophobic) attached to a core oligosaccharide and a distal polysaccharide. LPS binds to Toll-like receptor 4 (TLR4) and activates microglia (51) which in turn increases secretion of pro-inflammatory cytokines such as interleukin-6 (IL-6), tumor necrosis factor-α (TNF-α), and CXCL1/GROα. Two compounds were used as positive controls in this study:, TAK-242 (Resartovid), a TLR4 inhibitor, obtained from Invivogen and MCC950, a potent and specific inhibitor of the NLRP3 (NOD-like receptor pyrin domain-containing protein 3, cryopyrin, or NALP3) inflammasome (obtained from Invivogen).

Study Design

The drugs studied are listed in Table 9.

TABLE 9
Drugs studied in LPS/ATP induced neuroinflammation

| | | | Final Vehicle |
|---|---|---|---|
| | | Testing | nature and |
| Compound | Source | concentration | amount |
| Sodium | Bio-Techne | 500, 100 μM | 0.5% H2O |
| Phenylbutyrate | (Tocris) | | |
| Tauroursodeoxycholic | Sigma | 200, 40 μM | 0.08% H2O |
| acid | (Avanti) | | |
| Exenatide | Sigma | 100, 20 nM | Culture |
| | (Aldrich) | | medium |

Reagents

Control and Vehicle. The culture medium was BrainPhys™ Neuronal Medium, supplied by Stemcell Technologies, www.stemcell.com. Vehicles were culture medium alone, 0.5% and 0.08% H₂O in culture medium.

Lipopolysaccharide (LPS) (from E.coli 0111:B4; Invivogen reference tlrl-eblps) was prepared at a stock solution of 5 mg/mL in 100% water then diluted in culture medium.

Adenosine 5′-triphosphate (ATP) disodium salt (Invivogen; reference tlrl-atpl) was prepared at a stock solution of 725 mM in 100% water then diluted in culture medium.

Reference Substances

TAK-242, a TLR4 inhibitor (Resartovid, Invivogen Reference N° S7455) was prepared at a stock solution of 2.76 mM in 100% DMSO and then diluted in culture medium. TAK-242 (resatorvid), a small-molecule-specific inhibitor of Toll-like receptor (TLR) 4 signaling, inhibits the production of lipopolysaccharide (LPS)-induced inflammatory and proteolytic pathways (52).

MCC950, a NLRP3 inhibitor (Invivogen Reference N° inh-mcc) was prepared at a stock solution of 20 mM in 100% DMSO and then diluted in culture medium. MCC950 is an inhibitor of the NLRP3 inflammasome, a component of the inflammatory process (53).

Both TAK-242 and MCC950 are positive controls.

Test Substance Preparations

Sodium Phenylbutyrate, TUDCA, and exenatide were prepared in stock solutions and diluted to the concentrations noted above.

The selected doses were chosen based on the published literature: Sodium Phenylbutyrate=500 μM (10); Exenatide=100nM (54); TUDCA=200 μM (55), (56), (57). The combination of these 3 drugs was also evaluated for dose response at a 20% dose (100 μM, 20nM and 40 μM correspondingly).

Study Cells

Microglia cells were obtained from an iCell® Microglia kit, 01279 (Catalogue N° R1131) from FUJIFILM Cellular Dynamics. We followed the manufacturer's protocol for activation of the microglial cells with LPS/ATP to subsequently measure the effect of the study drugs on pro-inflammatory cytokines IL-6, TNF-α and CXCL1/GROα.

Experimental Procedure

All cell culture procedures were performed in aseptic conditions, under a laminar flow hood. Microglia were thawed and cultured following the provider's instructions. The microglia were plated at 50,000 cells per well of a Corning Primaria 96 well plate (Ref N° 353872) in 100 μL of growth medium. Cells were incubated at 37° C./5% CO₂ in a humidified cell culture incubator.

Three days after cell plating, the test or reference substances (25 μL of solutions at 5×) were applied 1 hour before LPS and ATP stimulation.

One hour later, 12.5 μL of test or reference substances were added again at 2× concentrated followed by 12.5 μL of LPS at 12× concentration. Fifty microliters of ATP were introduced at 4× concentration, 5 h 30 min after incubation with LPS.

Six hours after LPS incubation, the supernatant of each well was collected and centrifugated at 1000 g for 10 minutes to remove the cells and debris.

Fifty microliters of supernatant from NT and LPS+ATP-treated wells were used to determine cytokine secretion (IL-6, TNF-α, CXCL1/GROα) by Luminex platform according to the provider's instructions. Samples were diluted prior to the assay to fit within the standard range if needed. Three wells were tested per each condition (Table 10). Cytokines levels in the supernatant were expressed in pg/mL. The data was analyzed using a One-Way ANOVA followed by Bonferroni test.

TABLE 10
Experimental Combinations for Neuroinflammation Study Conditions

| | | | | Reference | |
|---|---|---|---|---|---|
| Compound | 1 | 2 | 3 | substances | Reagents |
| Control for | 0.5% H2O | 0.08% H2O | Medium | | Untreated cells |
| #1 to #9 | | | | | |
| Control for | 0.5% H2O | 0.08% H2O | Medium | | LPS (100 ng/mL) + ATP (5 mM) |
| #1 to #9 | | | | | |
| #1 | PBA (500 | 0.08% H2O | Medium | | LPS (100 ng/mL) + ATP (5 mM) |
| | μM) | | | | |
| #2 | 0.5% H2O | TUDCA (200 | Medium | | LPS (100 ng/mL) + ATP (5 mM) |
| | | μM) | | | |
| #3 | 0.5% H2O | 0.08% H2O | EXD (100 | | LPS (100 ng/mL) + ATP (5 mM) |
| | | | nM) | | |
| #4 | PBA (500 | 0.08% H2O | EXD (100 | | LPS (100 ng/mL) + ATP (5 mM) |
| | μM) | | nM) | | |
| #5 | PBA (500 | TUDCA (200 | Medium | | LPS (100 ng/mL) + ATP (5 mM) |
| | μM) | μM) | | | |
| #6 | 0.5% H2O | TUDCA (200 | EXD (100 | | LPS (100 ng/mL) + ATP (5 mM) |
| | | μM) | nM) | | |
| #7 | PBA (500 | TUDCA (200 | EXD (100 | | LPS (100 ng/mL) + ATP (5 mM) |
| | μM) | μM) | nM) | | |
| #8 | PBA (100 | TUDCA (40 | EXD (20 | | LPS (100 ng/mL) + ATP (5 mM) |
| | μM) | μM) | nM) | | |
| #9 | PBA (100 | 0.08% H2O | EXD (20 | | LPS (100 ng/mL) + ATP (5 mM) |
| | μM) | | nM) | | |
| Positive | | | | TAK-242 (2 | LPS (100 ng/mL) + ATP (5 mM) |
| control | | | | μM) | |
| Positive | | | | MCC950 (10 | LPS (100 ng/mL) + ATP (5 mM) |
| control | | | | μM) | |

TABLE 11
Tabulation of Study Conditions

| | Non-treated | | |
|---|---|---|---|
| Control/NT | cells | | |
| Control/LPS/ | Activated cells | | |
| ATP | | | |
| #1 | PBA (500 μM) | | |
| #2 | | TUDCA (200 μM) | |
| #3 | | | EXD (100 nM) |
| #4 | PBA (500 μM) | | EXD (100 nM) |
| #5 | PBA (500 μM) | TUDCA (200 μM) | |
| #6 | | TUDCA (200 μM) | EXD (100 nM) |
| #7 | PBA (500 μM) | TUDCA (200 μM) | EXD (100 nM) |
| #8 | PBA (100 μM) | TUDCA (40 μM) | EXD (20 nM) |
| #9 | PBA (100 μM) | | EXD (20 nM) |
| TAK-242 (2 | Positive | | |
| μM)/LPS/ATP | control | | |
| MCC950 (10 | Positive | | |
| μM)/LPS/ATP | control | | |

Results

TABLE 12.1
Inhibition of IL-6 mediated inflammation in microglial cells. (plotted in FIG. 8)

| Treatment | Replicate 1 | Replicate 2 | Replicate 3 | Mean ± S.D. |
|---|---|---|---|---|
| Control/NT | <10.47 | <10.47 | <10.47 | 10.47 ± 0.00 |
| Control/LPS/ATP | 761.03 | 628.90 | 686.39 | 692.11 ± 66.25 |
| #1 | 396.23 | 363.94 | 305.53 | 355.23 ± 45.97 |
| #2 | 410.97 | 442.87 | 459.60 | 437.81 ± 24.71 |
| #3 | 458.31 | 509.44 | 549.19 | 505.65 ± 45.56 |
| #4 | 173.06 | 201.13 | 235.63 | 203.27 ± 31.34 |
| #5 | 195.94 | 271.31 | 269.04 | 245.43 ± 42.87 |
| #6 | 416.59 | 399.70 | 289.38 | 368.56 ± 69.09 |
| #7 | 220.79 | 154.17 | 82.65 | 152.54 ± 69.08 |
| #8 | 243.92 | 186.93 | 81.05 | 170.63 ± 82.65 |
| #9 | 432.55 | 548.35 | 369.19 | 450.03 ± 90.85 |
| TAK-242 (2 μM) | <10.47 | <10.47 | <10.47 | 10.47 ± 0.00 |
| MCC950 (10 μM) | 253.54 | 297.91 | 218.00 | 256.48 ± 40.04 |

TABLE 12.2
Inhibition of TNF-α mediated neuroinflammation
in microglial cells (plotted in FIG. 9).

| Treatment | Replicate 1 | Replicate 2 | Replicate 3 | Mean ± S.D. |
|---|---|---|---|---|
| Control/NT | <5.63 | <5.63 | <5.63 | 5.63 ± 0.00 |
| Control/LPS/ATP | 53.21 | 61.30 | 64.70 | 59.74 ± 5.90 |
| #1 | 32.67 | 35.47 | 32.24 | 33.46 ± 1.75 |
| #2 | 29.01 | 27.92 | 28.79 | 28.57 ± 0.58 |
| #3 | 43.39 | 39.97 | 43.82 | 42.39 ± 2.11 |
| #4 | 21.85 | 23.59 | 27.06 | 24.17 ± 2.65 |
| #5 | 15.27 | 17.91 | 17.47 | 16.88 ± 1.41 |
| #6 | 32.02 | 28.79 | 22.72 | 27.84 ± 4.72 |
| #7 | 16.59 | 11.27 | <5.63 | 11.16 ± 5.48 |
| #8 | 23.15 | 19.88 | 12.83 | 18.62 ± 5.27 |
| #9 | 46.39 | 52.36 | 45.75 | 48.17 ± 3.65 |
| TAK-242 (2 μM)/ | <5.63 | <5.63 | <5.63 | 5.63 ± 0.00 |
| LPS/ATP | | | | |
| MCC950 (10 μM)/ | 29.44 | 29.87 | 33.53 | 30.95 ± 2.25 |
| LPS/ATP | | | | |

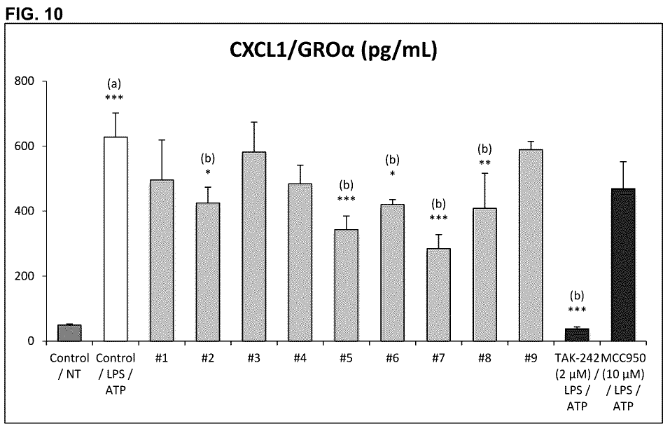

TABLE 12.3
Inhibition of CXCL1/GROα mediated neuroinflammation
in microglial cells (plotted in FIG. 10).

| Treatment | Replicate 1 | Replicate 2 | Replicate 3 | Mean ± S.D. |
|---|---|---|---|---|
| Control/NT | 50.56 | 52.17 | 45.24 | 49.32 ± 3.63 |
| Control/LPS/ATP | 668.57 | 672.54 | 543.08 | 628.06 ± 73.62 |
| #1 | 627.29 | 383.60 | 476.49 | 495.79 ± 122.99 |
| #2 | 447.19 | 368.84 | 458.86 | 424.96 ± 48.95 |
| #3 | 658.79 | 480.28 | 606.43 | 581.83 ± 91.76 |
| #4 | 478.60 | 428.97 | 544.17 | 483.91 ± 57.78 |
| #5 | 296.66 | 378.94 | 353.94 | 343.18 ± 42.18 |
| #6 | 404.76 | 435.79 | 419.92 | 420.16 ± 15.52 |
| #7 | 325.33 | 289.90 | 238.40 | 284.54 ± 43.71 |
| #8 | 503.84 | 431.45 | 291.69 | 408.99 ± 107.84 |
| #9 | 578.48 | 570.73 | 618.42 | 589.21 ± 25.59 |
| TAK-242 (2 μM)/ | 35.76 | 32.96 | 44.55 | 37.76 ± 6.05 |
| LPS/ATP | | | | |
| MCC950 (10 μM)/ | 375.70 | 499.15 | 533.31 | 469.39 ± 82.91 |
| LPS/ATP | | | | |

Discussion

Control—Inflammatory Cytokines: In the presence of LPS and ATP, the concentrations of IL-6, TNF-α, and GROα were significantly increased, as compared with non-treated conditions (IL-6: 529.7±108.13 pg/mL versus 10.47±0.00 pg/mL, p<0.001; TNF-α: 49.64±11.28 pg/mL versus 5.63±0.00 pg/mL, p<0.001; and GROα: 625.26±95.84 pg/mL versus 46.09±11.38 pg/mL, p<0.001).

Condition 1: PBA at 500 μM (#1) in presence of LPS and ATP significantly decreased the concentrations of IL-6 by 49% (p<0.001) and TNF-α by 44% (p<0.001), as compared with 0.58% distilled water in presence of LPS and ATP.

Condition 2: TUDCA at 200 μM (#2) in presence of LPS and ATP significantly decreased the concentrations of IL-6 by 37% (p<0.001), TNF-α by 52% (p<0.001) and to a lesser extent, GROα by 32% (p<0.05) as compared with 0.58% distilled water in presence of LPS and ATP.

Condition 3: EXD at 100 nM (#3) in presence of LPS and ATP decreased the concentrations of IL-6 by 27% (p<0.05) and TNF-α by 29% (p<0.001), as compared with 0.58% distilled water in presence of LPS and ATP.

Condition 4: PBA at 500 μM and EXD at 100 nM (#4) in presence of LPS and ATP decreased the concentrations of IL-6 by 71% (p<0.001), TNF-α by 60% (p<0.001) as compared with 0.58% distilled water in presence of LPS and ATP. The combination of the 2 treatments also significantly decreased the concentrations of IL-6 ( by 60%) and TNF-α (by 43%), as compared with EXD at 100 nM alone in presence of LPS and ATP (both p<0.001).

Condition 5: PBA at 500 μM and TUDCA at 200 μM (#5) in presence of LPS and ATP significantly decreased the concentrations of IL-6 by 65% (p<0.001), TNF-α by 72% (p<0.001) and GROα by 45% (p<0.001), as compared with 0.58% distilled water in presence of LPS and ATP. The combination of the 2 treatments also significantly decreased the concentrations of IL-6 by 44%, as compared with TUDCA at 200 μM alone in presence of LPS and ATP and the concentration of TNF-α by 50%, as compared with PBA at 500 μM alone in presence of LPS and ATP (p<0.01 for each cytokine).

Condition 6: TUDCA at 200 μM and EXD at 100 nM (#6) in presence of LPS and ATP significantly decreased the concentrations of IL-6 by 47% (p<0.001), TNF-α by 53% (p<0.001) and to a lesser extent GROα by 33% (p<0.05), as compared with 0.58% distilled water in presence of LPS and ATP. The combination of the 2 treatments also significantly decreased the concentration of TNF-α by 34%, as compared with EXD at 100 nM alone in presence of LPS and ATP (p<0.01).

Condition 7: PBA at 500 μM, TUDCA at 200 μM and EXD at 100 nM (#7) significantly decreased the concentrations of IL-6 by 78% (p<0.001), TNF-α by 81% (p<0.001) and GROα by 55% (p<0.001), as compared with 0.58% distilled water in presence of LPS and ATP. The combination of the 3 treatments also significantly decreased the concentrations of IL-6 by 57% and TNF-α by 67%, as compared with PBA at 500 μM alone (p<0.01 and p<0.001, respectively), 65% and 61% vs TUDCA at 200 μM alone (p<0.001 for both cytokines) and 70% and 74% vs EXD at 100 nM alone (p<0.001 for both cytokines) in presence of LPS and ATP. It also significantly decreased the concentration of GROα, as compared with PBA at 500 μM alone by 43% (p<0.05) and as compared with EXD at 100 nM alone by 51% (p<0.001) in presence of LPS and ATP.

Condition 8: Lower doses of PBA at 100 μM, TUDCA at 40 μM and EXD at 20 nM (#8) decreased the concentrations of IL-6 (p<0.001), TNF-α (p<0.001) and GROα (p<0.01), as compared with 0.58% distilled water in presence of LPS and ATP to lesser extent.

Condition 9: Lower doses of PBA at 100 μM and EXD at 20 μM (#9) in presence of LPS and ATP decreased the concentration of IL-6 (p<0.001), as compared with 0.58% distilled water in presence of LPS and ATP to lesser extent.

Additional combination remarks: PBA at 500 μM, TUDCA at 200 μM and EXD at 100 nM (#7) significantly decreased the concentrations of IL-6 by 30%, TNF-α by 58% (p<0.05) and GROα by 41% (p<0.05), as compared with a combination of PBA at 500 μM and EXD at 100 nM (#4); and decreased the concentrations of IL-6 by 42% (p<0.05), TNF-α by 40% (p<0.05) and GROα by 17%, as compared with a combination of PBA at 500 μM and TUDCA at 200 μM (#5), and decreased the concentrations of IL-6 by 61% (p<0.001), TNF-α by 63% (p<0.001) and GROα by 33% (p<0.01), as compared with a combination of TUDCA at 200 μM and EXD at 100 nM (#6).

Positive control TAK-242, a TLR4 inhibitor at 2 μM in presence of LPS and ATP significantly decreased the concentrations of IL-6 (p<0.001), TNF-α (p<0.001) and IL-1β (p<0.001) and GROα (p<0.001), as compared with 0.58% distilled water in presence of LPS and ATP.

Another positive control MCC-950, a NLRP3 inhibitor at 10 μM in presence of LPS and ATP significantly decreased the concentrations of IL-6 (p<0.001), TNF-α (p<0.001) and IL-1β (p<0.001), as compared with 0.58% distilled water in presence of LPS and ATP.

Conclusions

Anti-inflammatory effects of the individual drugs were observed with PBA at 500 μM, TUDCA at 200 μM and EXD at 100 nM. These effects were unexpectedly potentiated when the treatments were applied in different combinations. The most impressive, unexpected results were seen with a triple combination of PBA at 500 μM, TUDCA at 200 μM and EXD at 20 nM (#7) which was superior to all single and all double combinations and statistically significantly decreased levels of all three cytokines studied reducing the activation of the microglial cells, (FIG. 8, 9, 10). A combination of PBA and EXD (#4) also produced unexpected significant combinatory effect comparing to the individual drugs alone (PBA and EXD).

The effects were also observed with the combination of the three treatments at lower doses, i.e. PBA at 100 μM, TUDCA at 40 μM and EXD at 20 nM but were slightly lower suggesting dose response effect.

Because of the significance of neuroinflammation in dopaminergic neurodegeneration (FIG. 1), these results demonstrate promise in the treatment of synucleinopathic diseases.

Overall, this proves that the enhanced combinatory effect of the drugs is observed in two independent processes (dopaminergic degeneration and neuroinflammation) affecting two different cell lines (neurons and microglia) involved in neurodegenerative cascade seen in all alpha-synucleinopathies as shown in FIG. 1.

References
1. Rangel-Barajas C, Coronel I, Florán B. Dopamine Receptors and Neurodegeneration. Aging Dis. 2015;6(5):349-68.
2. Ni A, Ernst C. Evidence That Substantia Nigra Pars Compacta Dopaminergic Neurons Are Selectively Vulnerable to Oxidative Stress Because They Are Highly Metabolically Active. Frontiers in Cellular Neuroscience. 2022;16.
3. Pham C G, Bubici C, Zazzeroni F, Papa S, Jones J, Alvarez K, et al. Ferritin Heavy Chain Upregulation by NF-κB Inhibits TNFα-Induced Apoptosis by Suppressing Reactive Oxygen Species. Cell. 2004;119(4):529-42.
4. Dias V, Junn E, Mouradian M M. The role of oxidative stress in Parkinson's disease. Journal of Parkinson's disease. 2013;3(4):461-91.
5. Haining R, Achat-Mendes C. Neuromelanin, one of the most overlooked molecules in modern medicine, is not a spectator. Neural Regeneration Research. 2017;12(3):372.
6. Gerlach M, Double K L, Ben-Shachar D, Zecca L, Youdim M B H, Riederer P. Neuromelanin and its interaction with iron as a potential risk factor for dopaminergic neurodegeneration underlying Parkinson's disease. Neurotoxicity Research. 2003;5(1-2):35-43.
7. Viceconte N, Burguillos M A, Herrera A J, De Pablos R M, Joseph B, Venero J L. Neuromelanin activates proinflammatory microglia through a caspase-8-dependent mechanism. Journal of Neuroinflammation. 2015;12(1):5.
8. Singh N. The Role of Iron in Prion Disease and Other Neurodegenerative Diseases. PLoS Pathogens. 2014;10(9):e1004335.
9. López-Pérez Ó, Badiola J J, Bolea R, Ferrer I, Llorens F, Martin-Burriel I. An Update on Autophagy in Prion Diseases. Frontiers in Bioengineering and Biotechnology. 2020;8.
10. Roy A, Ghosh A, Jana A, Liu X, Brahmachari S, Gendelman H E, et al. Sodium phenylbutyrate controls neuroinflammatory and antioxidant activities and protects dopaminergic neurons in mouse models of Parkinson's disease. PLoS One. 2012;7(6):e38113.
11. Ono K, Ikemoto M, Kawarabayashi T, Ikeda M, Nishinakagawa T, Hosokawa M, et al. A chemical chaperone, sodium 4-phenylbutyric acid, attenuates the pathogenic potency in human alpha-synuclein A30P+A53T transgenic mice. Parkinsonism Relat Disord. 2009;15(9):649-54.
12. Truog P, inventor4-Phenylbutyric Acid Controlled-Release Formulations for Therapeutic Use patent US 2006/0045912 A1.
13. Cortez L, Sim V. The therapeutic potential of chemical chaperones in protein folding diseases. Prion. 2014;8(2).
14. Vang S, Longley K, Steer C J, Low W C. The Unexpected Uses of Urso- and Tauroursodeoxycholic Acid in the Treatment of Non-liver Diseases. Glob Adv Health Med. 2014;3(3):58-69.
15. Soto C, Pritzkow S. Protein misfolding, aggregation, and conformational strains in neurodegenerative diseases. Nat Neurosci. 2018;21(10):1332-40.
16. Cortez L M, Campeau J, Norman G, Kalayil M, Van der Merwe J, McKenzie D, et al. Bile Acids Reduce Prion Conversion, Reduce Neuronal Loss, and Prolong Male Survival in Models of Prion Disease. J Virol. 2015;89(15):7660-72.
17. Duan W M, Rodrigues C M, Zhao L R, Steer C J, Low W C. Tauroursodeoxycholic acid improves the survival and function of nigral transplants in a rat model of Parkinson's disease. Cell Transplant. 2002;11(3):195-205.
18. Weihe E, Depboylu C, Schutz B, Schafer M K, Eiden L E. Three types of tyrosine hydroxylase-positive CNS neurons distinguished by dopa decarboxylase and VMAT2 co-expression. Cell Mol Neurobiol. 2006;26(4-6):659-78.
19. Castro-Caldas M, Carvalho A N, Rodrigues E, Henderson C J, Wolf C R, Rodrigues C M, et al. Tauroursodeoxycholic acid prevents MPTP-induced dopaminergic cell death in a mouse model of Parkinson's disease. Mol Neurobiol. 2012;46(2):475-86.
20. Rosa A I, Fonseca I, Nunes M J, Moreira S, Rodrigues E, Carvalho A N, et al. Novel insights into the antioxidant role of tauroursodeoxycholic acid in experimental models of Parkinson's disease. Biochim Biophys Acta Mol Basis Dis. 2017;1863(9):2171-81.
21. Zangerolamo L, Vettorazzi J F, Rosa L R O, Carneiro E M, Barbosa H C L. The bile acid TUDCA and neurodegenerative disorders: An overview. Life Sci. 2021;272:119252.
22. Cai R, Zhang Y, Simmering J E, Schultz J L, Li Y, Fernandez-Carasa I, et al. Enhancing glycolysis attenuates Parkinson's disease progression in models and clinical databases. J Clin Invest. 2019;129(10):4539-49.
23. Simmering J E, Welsh M J, Liu L, Narayanan N S, Pottegard A. Association of Glycolysis-Enhancing alpha-1 Blockers With Risk of Developing Parkinson Disease. JAMA Neurol. 2021;78(4):407-13.
24. Kim S, Moon M, Park S. Exendin-4 protects dopaminergic neurons by inhibition of microglial activation and matrix metalloproteinase-3 expression in an animal model of Parkinson's disease. J Endocrinol. 2009;202(3):431-9.
25. Chen S, Yu S J, Li Y, Lecca D, Glotfelty E, Kim H K, et al. Post-treatment with PT302, a long-acting Exendin-4 sustained release formulation, reduces dopaminergic neurodegeneration in a 6-Hydroxydopamine rat model of Parkinson's disease. Sci Rep. 2018;8(1):10722.
26. Zhang L, Zhang L, Li L, Holscher C. Semaglutide is Neuroprotective and Reduces alpha-Synuclein Levels in the Chronic MPTP Mouse Model of Parkinson's Disease. J Parkinsons Dis. 2019;9(1):157-71.
27. Igoillo-Esteve M, Oliveira A F, Cosentino C, Fantuzzi F, Demarez C, Toivonen S, et al. Exenatide induces frataxin expression and improves mitochondrial function in Friedreich ataxia. JCI Insight. 2020;5(2).
28. Chang G, Liu J, Qin S, Jiang Y, Zhang P, Yu H, et al. Cardioprotection by exenatide: A novel mechanism via improving mitochondrial function involving the GLP-1 receptor/cAMP/PKA pathway. Int J Mol Med. 2018;41(3):1693-703.
29. Athauda D, Maclagan K, Skene SS, Bajwa-Joseph M, Letchford D, Chowdhury K, et al. Exenatide once weekly versus placebo in Parkinson's disease: a randomised, double-blind, placebo-controlled trial. Lancet. 2017;390(10103):1664-75.
30. Mishra A, Chandravanshi LP, Trigun SK, Krishnamurthy S. Ambroxol modulates 6-Hydroxydopamine-induced temporal reduction in Glucocerebrosidase (GCase) enzymatic activity and Parkinson's disease symptoms. Biochem Pharmacol. 2018;155:479-93.
31. Mullin S, Smith L, Lee K, D′Souza G, Woodgate P, Elflein J, et al. Ambroxol for the Treatment of Patients With Parkinson Disease With and Without Glucocerebrosidase Gene Mutations: A Nonrandomized, Noncontrolled Trial. JAMA Neurol. 2020;77(4):427-34.
32. Cabantchik Z I, Munnich A, Youdim M B, Devos D. Regional siderosis: a new challenge for iron chelation therapy. Front Pharmacol. 2013;4:167.
33. Devos D, Moreau C, Devedjian J C, Kluza J, Petrault M, Laloux C, et al. Targeting chelatable iron as a therapeutic modality in Parkinson's disease. Antioxid Redox Signal. 2014;21(2):195-210.
34. Burklen T S, Schlattner U, Homayouni R, Gough K, Rak M, Szeghalmi A, et al. The creatine kinase/creatine connection to Alzheimer's disease: CK-inactivation, APP-CK complexes and focal creatine deposits. J Biomed Biotechnol. 2006;2006(3):35936.
35. Investigators NN-P. A randomized, double-blind, futility clinical trial of creatine and minocycline in early Parkinson disease. Neurology. 2006;66(5):664-71.
36. Shults C W, Oakes D, Kieburtz K, Beal MF, Haas R, Plumb S, et al. Effects of coenzyme Q10 in early Parkinson disease: evidence of slowing of the functional decline. Arch Neurol. 2002;59(10):1541-50.
37. Beal M F, Shults C W. Effects of Coenzyme Q10 in Huntington's disease and early Parkinson's disease. Biofactors. 2003;18(1-4):153-61.
38. Yang L, Calingasan N Y, Wille E J, Cormier K, Smith K, Ferrante R J, et al. Combination therapy with coenzyme Q10 and creatine produces additive neuroprotective effects in models of Parkinson's and Huntington's diseases. J Neurochem. 2009;109(5):1427-39.
39. Hebron M L, Lonskaya I, Moussa C E. Nilotinib reverses loss of dopamine neurons and improves motor behavior via autophagic degradation of alpha-synuclein in Parkinson's disease models. Hum Mol Genet. 2013;22(16):3315-28.
40. Pagan F L, Hebron M L, Wilmarth B, Torres-Yaghi Y, Lawler A, Mundel E E, et al. Nilotinib Effects on Safety, Tolerability, and Potential Biomarkers in Parkinson Disease: A Phase 2 Randomized Clinical Trial. JAMA Neurol. 2020;77(3):309-17.
41. Pagan F L, Wilmarth B, Torres-Yaghi Y, Hebron ML, Mulki S, Ferrante D, et al. Long-Term Safety and Clinical Effects of Nilotinib in Parkinson's Disease. Mov Disord. 2021;36(3):740-9.
42. Peng J, Liu Q, Rao M S, Zeng X. Using human pluripotent stem cell-derived dopaminergic neurons to evaluate candidate Parkinson's disease therapeutic agents in MPP+ and rotenone models. J Biomol Screen. 2013;18(5):522-33.
43. Trudler D, Ghatak S, Lipton S A. Emerging hiPSC Models for Drug Discovery in Neurodegenerative Diseases. Int J Mol Sci. 2021;22(15).
44. Caserta T M, Smith A N, Gultice A D, Reedy M A, Brown T L. Q-VD-OPh, a broad spectrum caspase inhibitor with potent antiapoptotic properties. Apoptosis. 2003;8(4):345-52.
45. Baden M Y, Fukui K, Hosokawa Y, Iwahashi H, Imagawa A, Shimomura I. Examination of a Viral Infection Mimetic Model in Human iPS Cell-Derived Insulin-Producing Cells and the Anti-Apoptotic Effect of GLP-1 Analogue. PLoS One. 2015;10(12):e0144606.
46. Magalhaes J, Gegg M E, Migdalska-Richards A, Schapira A H. Effects of ambroxol on the autophagy-lysosome pathway and mitochondria in primary cortical neurons. Sci Rep. 2018;8(1):1385.
47. Aguirre P, Mena NP, Carrasco C M, Munoz Y, Perez-Henriquez P, Morales R A, et al. Iron Chelators and Antioxidants Regenerate Neuritic Tree and Nigrostriatal Fibers of MPP+/MPTP-Lesioned Dopaminergic Neurons. PLoS One. 2015;10(12):e0144848.
48. Cunha M P, Martin-de-Saavedra M D, Romero A, Parada E, Egea J, Del Barrio L, et al. Protective effect of creatine against 6-hydroxydopamine-induced cell death in human neuroblastoma SH-SY5Y cells: Involvement of intracellular signaling pathways. Neuroscience. 2013;238:185-94.
49. Cooper O, Seo H, Andrabi S, Guardia-Laguarta C, Graziotto J, Sundberg M, et al. Pharmacological rescue of mitochondrial deficits in iPSC-derived neural cells from patients with familial Parkinson's disease. Sci Transl Med. 2012;4(141):141ra90.
50. Doherty K R, Wappel R L, Talbert D R, Trusk P B, Moran D M, Kramer J W, et al. Multi-parameter in vitro toxicity testing of crizotinib, sunitinib, erlotinib, and nilotinib in human cardiomyocytes. Toxicol Appl Pharmacol. 2013;272(1):245-55.
51. Bassil R, Shields K, Granger K, Zein I, Ng S, Chih B. Improved modeling of human AD with an automated culturing platform for iPSC neurons, astrocytes and microglia. Nat Commun. 2021;12(1):5220.
52. Ono Y, Maejima Y, Saito M, Sakamoto K, Horita S, Shimomura K, et al. TAK-242, a specific inhibitor of Toll-like receptor 4 signalling, prevents endotoxemia-induced skeletal muscle wasting in mice. Sci Rep. 2020;10(1):694.
53. Coll R C, Robertson A A, Chae J J, Higgins S C, Munoz-Planillo R, Inserra M C, et al. A small-molecule inhibitor of the NLRP3 inflammasome for the treatment of inflammatory diseases. Nat Med. 2015;21(3):248-55.
54. Gullo F, Ceriani M, D′Aloia A, Wanke E, Constanti A, Costa B, et al. Plant Polyphenols and Exendin-4 Prevent Hyperactivity and TNF-alpha Release in LPS-Treated In vitro Neuron/Astrocyte/Microglial Networks. Front Neurosci. 2017;11:500.
55. Romero-Ramirez L, Garcia-Rama C, Wu S, Mey J. Bile acids attenuate PKM2 pathway activation in proinflammatory microglia. Sci Rep. 2022;12(1):1459.
56. Yanguas-Casas N, Barreda-Manso MA, Nieto-Sampedro M, Romero-Ramirez L. TUDCA: An Agonist of the Bile Acid Receptor GPBAR1/TGRS With Anti-Inflammatory Effects in Microglial Cells. J Cell Physiol. 2017;232(8):2231-45.
57. Kim S J, Ko W K, Jo M J, Arai Y, Choi H, Kumar H, et al. Anti-inflammatory effect of Tauroursodeoxycholic acid in RAW 264.7 macrophages, Bone marrow-derived macrophages, BV2 microglial cells, and spinal cord injury. Sci Rep. 2018;8(1):3176.

1. A method of treating a neurodegenerative disorder in a patient at risk for a neurodegenerative disorder associated with misfolding of α-synuclein or having symptoms of a neurodegenerative disorder associated with misfolding of α-synuclein, comprising the administration of a combination of two or more drugs selected from a chemical chaperone class of drugs, a glycolysis enhancer class of drugs, a glucagon-like-peptide-1 agonist (GLP-1) class of drugs, a glucocerebrosidase (GCase) inducer class of drugs, an iron chelator class of drugs, a mitochondrial antioxidant class of drugs, a cluster-Abelson (c-Abl) tyrosine kinase inhibitor class of drugs and a bile acid class of drugs.

2. The method of claim 1, wherein the combination comprises three or more drugs selected from a chemical chaperone class of drugs, a glycolysis enhancer class of drugs, a glucagon-like-peptide-1 agonist (GLP-1) class of drugs, a glucocerebrosidase (GCase) inducer class of drugs, an iron chelator class of drugs, a mitochondrial antioxidant class of drugs, a cluster-Abelson (c-Abl) tyrosine kinase inhibitor class of drugs and a bile acid class of drugs.

3. The method of claim 1, wherein
a. the chemical chaperone class of drugs comprises one or more of Sodium Phenylbutyrate (PBA) and arimoclomol;
b. the bile acid class of drugs comprises one or more of tauroursodeoxycholic acid (TUDCA), ursodeoxycholic acid (UDCA) and deoxycholic acid (DCA);
c. wherein the glycolysis enhancer comprises terazosin (TZ)
d. wherein the GLP-1 agonist class of drugs comprises one or more of Exenatide, ORMD-0901, dulaglutide, semaglutide, liraglutide, or lixisenatide;
e. wherein the GCase inducer class of drugs comprises ambroxol (AMB), BIA 28-5156 (LTI-291); isofagomine; LB-205 and S-181;
f. wherein the iron chelator class of drugs comprises a drug selected from deferiprone (DFP), deferoxamine (DFO), desferrioxamine, deferasirox, clioquinol, tetrahydrosalen, 5,7-Dichloro-2-[(dimethylamino) methyl]quinolin-8-ol (PBT2), (N,N,N,N-Tetrakis(2-pyridylmethyl)-ethylenedi-amine) (TPEN), 1,10-phenanthroline (PHEN), 1,2-hydroxypyridinone (1,2-HOPO), clioquinol; 5-[N-methyl-N-propargylaminomethyl]-8-hydroxyquinoline dihydrochloride (M30); M31; M32; -[4-(2-hydroxyethyl)piperazine-1-ylmethyl]-quinoline-8-ol] (VK28), HLA16, HLA20, M32, M10, SIH-B, BSIH, pyridoxal isonicotinoyl hydrazine (PIH); 2-pyridylcarboxaldehyde isonicotinoyl hydrazine (PCIH), H₂NPH, and H₂PPH;
g. wherein the mitochondrial antioxidant class of drugs comprises creatinine or CoQ10;
h. wherein the c-Abl tyrosine kinase inhibitor class of drugs comprises a drug selected from nilotinib radotinib, vodobatinib (K0706), bafetinib, imatinib, dasatinib, bosutinib, ponatinib, rebastinib, tozasertib, and danusertib

4. The method of claim 3 wherein PBA is provided in an extended-release formulation.

5. The method of claim 1, wherein the neurogenerative disorder is an alpha-synucleinopathy selected from Parkinson's disease (PD), Diffuse Lewy body Disease (DLBD) and Multiple System Atrophy (MSA).

6. A method of treating a neurodegenerative disorder associated with misfolding of α-synuclein comprising the administration, to a patient at risk for a neurodegenerative disorder or having symptoms of a neurodegenerative disorder, comprising the administration of a combination of two or more drugs selected from sodium phenylbutyrate (PBA), tauroursodeoxycholic acid (TUDCA), exenatide (EXD), deferiprone (DFP), terazosin (TZ), creatine (CR), CoQ10, Ambroxol (AMB), and nilotinib (NL) or dasatinib (DS).

7. The method of claim 6 wherein the combination comprises three or more drugs selected from sodium phenylbutyrate (PBA), tauroursodeoxycholic acid (TUDCA), exenatide, deferiprone (DFP), terazosin (TZ), Creatine, CoQ10, Ambroxol (AMB), and nilotinib (NL or dasatinib (DS).

8. The method of claim 6 wherein the combination comprises sodium phenylbutyrate (PBA) and exenatide.

9. The method of claim 6 wherein the combination comprises sodium phenylbutyrate (PBA), tauroursodeoxycholic acid (TUDCA), and exenatide (EXD).

10. The method of claim 6 wherein the combination comprises NL/DS and TUDCA.

11. The method of claim 6 wherein the combination comprises PBA, CR, and CoQ10.

12. The method of claim 6 wherein the combination comprises EXD and TUDCA.

13. The method of claim 6 wherein the combination comprises PBA, EXD, and AMB.

14. The method of claim 6 wherein the combination comprises EXD, NL/DS, and TUDCA.

15. The method of claim 6 wherein the combination comprises PBA, EXD, DFP.

16. The method of claim 6 wherein the combination comprises PBA, NL/DS, and TUDCA.

17. The method of claim 6 wherein the combination comprises EXD and NL/DS.

18. The method of claim 6 wherein the combination comprises PBA, EXD, and NL/DS.

19. The method of claim 6 wherein the combination comprises PBA, EXD, TUDCA, and DFP.

20. The method of claim 6 wherein the neurodegenerative disorder is Multiple System Atrophy (MSA) and the drugs administered are sodium phenylbutyrate (PBA) and tauroursodeoxycholic acid (TUDCA).
